(19) United States
(12) Patent Application Publication
(10) Pub. No.: US 2025/0020634 A1
(43) Pub. Date: Jan. 16, 2025

(54) METHOD TO INVESTIGATE FUNCTION OF VOLTAGE-GATED SODIUM CHANNELS IN CELLS

(71) Applicant: UNIVERSITY OF IOWA RESEARCH FOUNDATION, Iowa City, IA (US)

(72) Inventors: Christopher AHERN, Iowa City, IA (US); Robert PIPER, Iowa City, IA (US); Annabel MINARD, Iowa City, IA (US); Colin CLARK, Iowa City, IA (US)

(73) Assignee: UNIVERSITY OF IOWA RESEARCH FOUNDATION, Iowa City, IA (US)

(21) Appl. No.: 18/756,908

(22) Filed: Jun. 27, 2024

Related U.S. Application Data
(60) Provisional application No. 63/525,667, filed on Jul. 8, 2023.

(51) Int. Cl. G01N 33/50; C12N 5/077; G01N 33/68

(52) U.S. Cl. CPC G01N 33/5041; C12N 5/0656; G01N 33/6872; G01N 2333/46

(57) ABSTRACT

In a first aspect, provided herein is a beta/MPZ family-subunit-eliminated (beta/MPZΔ) engineered-cell comprising a cell with functionally inactivated SCN1B, SCN2B, SCN3B, SCN4B, MPZL1, MPZL2, MPZL3, MPZ and JAML genes. In certain aspects, the beta/MPZΔ cell further comprising a defined engineered voltage-gated sodium channel, wherein the engineered voltage-gated sodium channel comprises a sodium channel alpha subunit, at least one sodium channel beta subunit.

In certain aspect, provided herein is a method of screening effectiveness of a drug targeting a voltage-gated sodium channel (VGSC) disease or disorder, comprising:
(a) culturing a beta/MPZ family-subunit-eliminated (beta/MPZΔ) engineered-cell comprising a cell with functionally inactivated SCN1B, SCN2B, SCN3B, SCN4B, MPZL1, MPZL2, MPZL3, MPZ and JAML genes, and comprising a defined engineered voltage-gated sodium channel, wherein the engineered voltage-gated sodium channel comprises a sodium channel alpha subunit, at least one sodium channel beta subunit;
(b) introducing a target drug to the cells;
(c) applying a high-throughput patch-clamping to the cells to measure gating properties of the cells in the presence of the drugs as compared to control cells; and
(d) comparing the gating properties of the beta/MPZΔ cell the gating properties of the controls cell.

### CROSS REFERENCE TO RELATE APPLICATION RESEARCH

This application claims priority to U.S. Provisional Application No. 63/525,667 that was filed on Jul. 8, 2023. The entire content of the applications referenced above is hereby incorporated by reference herein.

### STATEMENT REGARDING FEDERALLY SPONSORED RESEARCH

This invention was made with government support under GM106568 and GM106568awarded by National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 9, 2024, is named 17023_274US1_SL.xml and is 72,679 bytes in size.

### BACKGROUND

Voltage-gated ion channels (VGICs) are transmembrane proteins that play important roles in the electrical signaling of cells. The activity of VGICs is regulated by the membrane potential of a cell, and open channels allow the movement of ions along an electrochemical gradient across cellular membranes. Depending on the ions conducted, VGICs can be classified as voltage-gated sodium, potassium, calcium, or chloride channels. Voltage-gated sodium (Na_{V}) channels, which run every electrical current in the body, have two subunits: alpha and beta.

The Voltage-gated sodium channel (VGSCs) complex is comprised of a large pore-forming a subunit that is partnered with β-subunits (β1-4), which are thought to regulate every aspect of the channel including trafficking, degradation, voltage-dependent functions, and pharmacology. Defects in VGSC function resulting from disease remodeling or inherited mutations in either the a or β subunits are established causes of human disease associated with sudden infant death, atrial fibrillation, reperfusion and ischemia injury, arrhythmia in the failing heart, epilepsy, and a variety of pain-causing syndromes.

It has been difficult to study the effect of the beta subunits in human cells because human cells express endogenous beta subunits. The molecular bases for these wide-ranging effects are poorly resolved primarily because the “gold standard” heterologous cells that are used for ion channel characterization and high-throughput screening exhibit near ubiquitous expression of β-subunits, and their near relatives. Toad oocytes, which lack voltage-gated sodium (Na_{V}) β-subunits, have been used historically to fill this gap, but these cells have poor homology in their membrane composition (lipid/cholesterol), display aberrant pharmacology due to their large size and lipidic cytoplasm, and are not amenable to high-throughput assays.

In the absence of a “clean” expression system, the specific functions of particular a subunits on VGSC function and the electrophysiological consequences of disease-causing mutations within them remain unresolved. This roadblock also hinders development of rational clinical approaches for effective therapies. In particular, there is a lack of a platform for drug-discovery that can precisely monitor the effect of small molecules on the gating properties of defined VGSC α/β complexes that would otherwise lead to drugs with greater specificity. This hampers the ability to screen for drugs that specifically modify the beta subunit regulation. Such drugs could be a whole new class of therapeutics. Accordingly, a novel cell line is needed that lacks all beta subunit proteins. There is also a need to establish a platform that allows high-throughput assessment of individual VGSC subunit functions to (1) expand the knowledge of how defects in these individual components can lead to cardiac dysfunction and disease, and (2) to develop new drugs that can selectively alter the behavior of specific VGSCs.

### SUMMARY

In a first aspect, provided herein is a beta/MPZ family-subunit-eliminated (beta/MPZΔ) engineered-cell comprising a cell with functionally inactivated SCN1B, SON2B, SON3B, SCN4B, MPZL1, MPZL2, MPZL3, MPZ and JAML genes. In certain aspects, the beta/MPZΔ cell further comprising a defined engineered voltage-gated sodium channel, wherein the engineered voltage-gated sodium channel comprises a sodium channel alpha subunit, at least one sodium channel beta subunit.

In certain aspect, provided herein is a method of screening effectiveness of a drug targeting a voltage-gated sodium channel (VGSC) disease or disorder, comprising:
(a) culturing a beta/MPZ family-subunit-eliminated (beta/MPZΔ) engineered-cell comprising a cell with functionally inactivated SCN1B, SON2B, SON3B, SON4B, MPZL1, MPZL2, MPZL3, MPZ and JAML genes, and comprising a defined engineered voltage-gated sodium channel, wherein the engineered voltage-gated sodium channel comprises a sodium channel alpha subunit, at least one sodium channel beta subunit;
(b) introducing a target drug to the cells;
(c) applying a high-throughput patch-clamping to the cells to measure gating properties of the cells in the presence of the drugs as compared to control cells; and
(d) comparing the gating properties of the beta/MPZΔ cell the gating properties of the control cell.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1E. Generation of BeHAPe cells by two rounds of CRISPR-Cas9 deletion of β-subunits and their phylogenetic relatives. (FIG. 1A) BeHAPe cells were engineered from eHAP-FRT cells, which have an FRT integration site between GPR108 and TRIP10. EGFP-NeoR was originally expressed from this locus but deleted in the second round of CRISPR-Cas9 deletions. (FIG. 1B) eHAP-FRT cells were engineered into BeHAPe cells with two rounds of CRISPR-Cas9. Multiple gRNA, Cas9 and fluorescent marker encoding plasmids were transfected into the eHAP-FRT cell-line. Transfected cells were enriched for by flow-sorting for fluorescent cells and serially diluted onto 10 cm dishes. Clones were screened to verify haploidy and then genotyped. (FIG. 1C) BeHAPe cells were deleted of the genes marked with a cross, a single-line indicates the loci was edited but not necessarily with a frame-shifting mutation. Genes disrupted in either round 1 or round 2 are indicated. (FIG. 1D) Residues that remain or were deleted in the mutant allele are colored black or red, respectively. Protein sequence was visualized with Protter. (FIG. 1E) N-terminus of MPZ which was deleted includes a β-strand in the Ig domain (PDB: 30AI). Deleted region is highlighted red.

FIGS. 2A-2B. FIGS. 2A and 2B together show mutations in β-subunits and their phylogenetic relatives in BeHAPe cells. Exons are drawn as purple boxes, deleted regions are colored green, while insertions are colored cyan. gRNA binding sites are indicated with a black and red bar, red bar indicates PAM sequence. The position of gene loci is derived from Genome Reference Consortium Human Build 38. Figure discloses SEQ ID NOS 1-11, respectively, in order of appearance.

FIGS. 3A-3E. Preparation of BeHAPe cells for electrophysiology. (FIG. 3A) BeHAPe cells were gene-edited while haploid. Diploids were used for electrophysiology experiments. Haploid population of BeHAPe cells become spontaneously diploid after several passages, and diploids can be enriched by cell sorting. (FIG. 3B) Haploid or diploid BeHAPe cells were selected for by flow sorting based on cell size. BeHAPe cells were stained with Hoechst 33342 and DNA content was compared to forward (FSC) and side (SSC) scatter by flow cytometry. Micrograph of haploid and diploid cells are shown. (FIG. 3C) Diploid BeHAPe cells were prepared for electrophysiology as shown. Cells were “reverse”-transfected and 24 hours later reseeded as single cells. To obtain healthy single cells, cells were lifted with versene, resuspend in conditioned media, passed through a 10 μm mesh, and dissociated further with gentle pipetting. 36-48 h after transfection cells were phenotyped with electrophysiology. (FIG. 3D) BeHAPe cell membranes became blebby when resuspended in fresh media, however, remained smooth in conditioned media. (FIG. 3E) Sodium current recordings from BeHAPe cells un-transfected or transfected with β1 or Na_{V}1.5 cDNA, as indicated.

FIG. 4. Biophysical properties of Na_{V}1.5 expressed in eHAP vs BeHAPe cells in the presence and absence of β1. The voltage dependence on the rate of inactivation (Top), the voltage dependence of activation (GV) (middle), and steady-state inactivation (SSI) curves (bottom) were determined for NaVNa_{V}1.5 expressed in eHAP cells and BeHAPe cells in the presence and absence of co-expression with β1 (SCN1B).

FIGS. 5A-5F. Biophysical properties of Na_{V}1.5 when co-expressed with β/MPZ family members. Plots for Na_{V}1.5 alone are identical across graphs, n=10. (FIG. 5A) Na_{V}1.5 co-expressed with rat β1-V5-6×HIS shifts GV V_{1/2}+7.5±0.6 mV, and SSI V_{1/2}+7.5±0.4 mV (Na_{V}1.5+b1 n=9). (FIG. 5B) Na_{V}1.5 co-expressed with rat b2-HA does not shift GV V_{1/2} but shifts SSI V_{1/2}−5.9±0.5 mV (Na_{V}1.5+β2 n−6). (FIG. 5C) Na_{V}1.5 co-expressed with human b3 shifts the GV V_{1/2}+7.4±0.9 mV and SSI V_{1/2}+9.4±0.6 mV (Na_{V}1.5+β3 n=6). (FIG. 5D) Na_{V}1.5 co-expressed with human b4 shifts GV V_{1/2}+6.1±0.6 mV but not SSI V_{1/2} (Na_{V}1.5+β4 n=6). (FIG. 5E) Na_{V}1.5 simultaneously co-expressed with b1 and b2 shifts GV V_{1/2}+9.3±0.6 mV and SSI V_{1/2}+13.4±0.5 mV (Na_{V}1.5+β1±β2 n=7). (FIG. 5F) Na_{V}1.5 co-expressed with MPZ shifts, GV V_{1/2}+7.4±0.6 mV and SSI V_{1/2}+6.9±0.3 mV (Na_{V}1.5+MPZ n=6). Data are plotted as the mean±SD. *p<0.05, **p<0.01 by one-way ANOVA with Dunnet correction for multiple comparisons. Fitted lines were generated by fitting a Boltzmann function to mean values.

FIGS. 6A-6F. B/MPZ proteins effects on Na_{V}1.5 inactivation kinetics. Plots for Na_{V}1.5 alone are identical across graphs, n=8. (FIG. 6A) Co-expression with β1 significantly speeds the rate of Na_{V}1.5 inactivation across all analyzed voltages (Na_{V}1.5+β1 n=8). (FIG. 6B) β2 does not alter Na_{V}1.5 inactivation rates (Na_{V}1.5+β2 n=6). (FIG. 6C) β3 slows Na_{V}1.5 inactivation at more negative potentials (Na_{V}1.5+β3 n=6). (FIG. 6D) 34 does not alter Na_{V}1.5 inactivation rates (Na_{V}1.5+β4 n=6). (FIG. 6E) Simultaneous co-expression with β1 and β2 does not alter Na_{V}1.5 inactivation rates (Na_{V}1.5+β1+β2 n=7). (FIG. 6F) MPZ slows Na_{V}1.5 inactivation rates (Na_{V}1.5+MPZ n=6). Data are plotted as the mean±SD. *p<0.05, **p<0.01 by one-way ANOVA with Dunnet correction for multiple comparisons. Rates were derived from a single exponential function manually fit to individual traces.

FIG. 7. β1 modulation of Na_{V}1.5 measured in BeHAPe and other systems. Electrophysiological properties previously measured in the indicated expression systems (8, 9, 11, 40, 42-46, 49) were plotted as the difference (Δ) between Na_{V}1.5 alone and Na_{V}1.5 in the presence of β1 co-expression. Values reported in Table 1 & 2 are plotted here.

FIGS. 8A-8E. Plasmids and oligonucleotides that were used in this study. Figure discloses SEQ ID NOS 12, 12-13, 13, 10, 14-15, 15-43, 43, 43, 43, 43, 43, 43-47, 47-48, 48, 48, 48-51, 51, 51, 51, 51, 51, 51-55, 55-56, 56, 56, 56-57, 50, 43, 51, 58-65, 55, 66, 56, 67-81 and 78, respectively, in order of columns.

### DETAILED DESCRIPTION

The method described herein is used to make cells that in turn provide a platform for small molecular screening and validation to find drugs that modify specific properties of defined α/β VGSC complexes. For instance, a major contributor to heart arrhythmia and failure is the persistence of a late sodium current (I_{Na}^{late}) caused by altered inactivation kinetics, yet there is a paucity of drugs that can effectively and selectively block late current, and thus this remains a significant untapped therapeutic avenue for clinical management of cardiac remodeling. The present data show that in the absence of β subunits, VGSC inactivation is very slow and greatly accelerated with different kinetics depending on the complement of particular β subunits. Thus, screening for drugs that can inhibit I_{Na}^{late} need to take place in a system where defined Na_{V} α/β complexes can be controlled. Moreover, screening defined α/β VGSC complexes has the potential to identify allosteric modifiers of β subunit unit function as well as β-specific modulators that might target particular α/β VGSC complexes that might deconvolute the interplay of different channel types and their contribution to heart disease.

Such cell lines also provide a preclinical pharmacological safety drug-screening tool to evaluate effects on the electrophysiological function of defined α/β VGSC complexes. In vitro drug safety screening is an essential component in developing new drugs. Employing the discovery platform developed under this proposal would complement existing in vitro cardiac safety testing programs by providing the means to test for off-target effects on cardiac-relevant neurological α/β VGSC complexes.

The present invention provides a method to remove multiple genes from a cell, allowing that cell to be a model system to analyze the function of voltage gated sodium channels for both basic biology as well as for drug screening. In one aspect, a cell line was engineered that lacks all beta subunit proteins. It was discovered that this novel cell line unexpectedly possessed new emergent properties of the heart voltage-gated sodium channel. It was not known until these novel cells were engineered that elimination of all of these proteins would allow new properties of voltage-gated sodium channels to be observed.

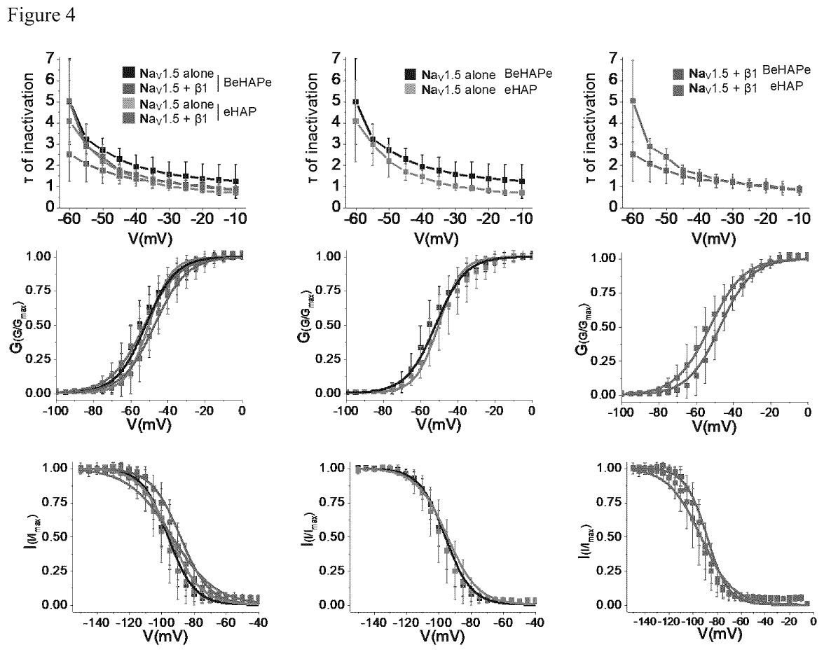

In certain aspects, the present invention involves eliminating a whole family of beta subunits that would otherwise interfere with the ability to discern what each individual beta subunit was doing to a voltage-gated sodium channel. FIG. 4 shows the cell line made by the inventors as compared to the starting cell line. The data show that the behavior of the Na_{V}1.5 sodium channel is different and that the effects of co-expressing a beta subunit are different between the two cell lines. Namely, the effect of exogenous beta subunit expression is masked in the parental cell line, but unveiled in the cell line that was made that eliminates the whole beta/MPZ family of proteins. Thus, these data demonstrate that eliminating endogenous β/MPZ family in BeHAPe cells sensitized this cell line to β1 effects, and thus is consistent with the proposition that eliminating endogenous β-subunits improves the detection of exogenous β-subunit effects.

### Voltage-Gated Sodium Channel (VGSC) Proteins

The Na_{V} channels are composed of one a subunit, which can be coupled to one or two β subunits.

In humans there are at least nine different a subunits: Na_{V}1.1, Na_{V}1.2, Na_{V}1.3, Na_{V}1.4, Na_{V}1.5, Na_{V}1.6, Na_{V}1.7, Na_{V}1.8, and Na_{V}1.9, which are encoded by the genes SCN1A, SCN2A, SCN3A, SCN4A, SCN5A, SCN8A, SCN9A, SCN10A, and SCN11A, respectively.

The different α subunits define the distinct Na_{V} channel subtypes and contain the receptor sites for drugs and toxins that act on Nav channels. The α subunits are large, single-chain polypeptides composed of approximately 2000 amino acid residues organized in four homologous domains, designated DI to DIV, that form a pseudo-tetrameric structure. The four domains are connected by intracellular linkers. Each domain contains six transmembrane helical segments (S1-S6), where segments S1-S4 form the voltage-sensing domain (VSD). Segments S5 and S6, and the connecting pore-loops (P-loops) form the channel pore. The intracellular loop connecting DIIIS6 and DIVS1 functions as an inactivation gate, closing the channel pore during fast inactivation. The sequence homology of mammalian Na_{V}subtypes is very high, being greater than 50% in the transmembrane and extracellular domains. Each domain is composed of six transmembrane helical segments named S1 to S6.

There are four voltage-gated sodium channel beta subunit genes, SCN1B, SCN2B, SCN3B, and SCN4B, encoding five proteins, β1, β1B, β2, β3, and β4. Subunits β1 and β1B are splice isoforms encoded by the SCN1B gene. Subunits β1, β2, β3, and β4 contain an extracellular Ig domain, one transmembrane domain, and a regulatory intracellular domain with multiple phosphorylation sites and secretase cleavage sites. While the β subunits are not pore-forming, they regulate VGSC localization and kinetics as well as participate in cell-cell adhesion and regulated intramembrane proteolysis as immunoglobulin (Ig) superfamily cell adhesion molecules (CAMs).

Subunits β2 or β4 bind to the a subunit via a disulfide bond, whereas β1 and β3 subunits are associated noncovalently. All β subunits are transmembrane proteins, except β1B, which is expressed as a soluble molecule. Although the a subunit alone is sufficient to form a fully functional Na_{V} channel, β subunits play crucial roles in the fine-tuning of channel kinetics and channel expression on the cell surface. Na_{V}β subunits are members of the immunoglobulin (Ig) superfamily of cell adhesion molecules (CAMs) possessing an extracellular Ig domain that participates in a number of cell adhesion-related activities.

Mutations in the genes encoding the Na_{V}α subunits, and the β subunits can affect functional channel expression or alter gating properties of these channels. As a consequence, mutations can lead to channel dysfunctions, giving rise to abnormal neuronal firing and associated disease phenotypes called channelopathies.

### Myelin Protein Zero (MPZ) Family

Myelin protein zero (MPZ, P0) is a single membrane glycoprotein, which in humans is encoded by the MPZ gene. The MPZ gene is specifically expressed in Schwann cells of the peripheral nervous system and encodes a type I transmembrane glycoprotein that is a major structural protein of the peripheral myelin sheath. The encoded protein contains a large hydrophobic extracellular domain and a smaller basic intracellular domain, which are essential for the formation and stabilization of the multilamellar structure of the compact myelin. Mutations in this gene are associated with autosomal dominant form of Charcot-Marie-Tooth disease type 1 (CMT1B) and other polyneuropathies, such as Dejerine-Sottas syndrome (DSS) and congenital hypomyelinating neuropathy (CHN). The IG domain of MPZ closely resembles that found in sodium channel β subunits.

The myelin protein zero like 1 (MPZL1) gene is predicted to enable structural molecule activity. It is located in the cell surface and functions in focal adhesion. It is predicted to be involved in cell-cell signaling and transmembrane receptor protein tyrosine kinase signaling pathway. It is further predicted to act upstream of or within positive regulation of cell migration.

The myelin protein zero like 2 (MPZL2) gene is expressed in the thymus and in several epithelial structures early in embryogenesis. It is highly homologous to the myelin protein zero and, in thymus-derived epithelial cell lines, is poorly soluble in nonionic detergents, strongly suggesting an association to the cytoskeleton. Its capacity to mediate cell adhesion through a homophilic interaction and its selective regulation by T cell maturation might imply the participation of EVA in the earliest phases of thymus organogenesis. The protein bears a characteristic V-type domain and two potential N-glycosylation sites in the extracellular domain; a putative serine phosphorylation site for casein kinase 2 is also present in the cytoplasmic tail. Two transcript variants encoding the same protein have been found for this gene.

The myelin protein zero like 3 (MPZL3) gene is predicted to be involved in cell adhesion and to be active in the plasma membrane. It is also predicted to be integral component of membrane. It is further predicted to act upstream of or within extracellular matrix organization and hair cycle.

### Junctional Adhesion Molecule-Like (JAML) Protein

Junctional Adhesion Molecule-Like (JAML) protein contains 2 extracellular immunoglobulin-like domains, a transmembrane segment, and a cytoplasmic tail. It is a member of the immunoglobulin superfamily, among novel retinoic acid-induced genes identified in acute promyelocytic leukemia (APL) cells. JAML protein is localized at the cell plasma membrane in the areas of cell-cell contacts, whereas it is not detected at free cell borders, suggests that JAML is engaged in homophilic interactions. Furthermore, a conserved dimerization motif among JAM members was shown to be important for JAML localization at the cell membrane.

### Cell Lines

In certain aspects, provided herein is a beta/MPZ family-subunit-eliminated (beta/MPZΔ) engineered-cell comprising a cell with functionally inactivated SCN1B, SON2B, SCN3B, SCN4B, MPZL1, MPZL2, MPZL3, MPZ and JAML genes. These cells may also be named “beta-subunit-eliminated haploid cells for expression” (BeHAPe) cells.

In certain aspects, the inactivating mutations are stable mutations.

In certain aspects, all copies these genes are mutated.

In certain aspects, the cell is devoid of normal SCN1B, SON2B, SCN3B, SCN4B, MPZL1, MPZL2, MPZL3, MPZ and JAML expression. In other words, the expression levels of the SCN1B, SCN2B, SCN3B, SCN4B, MPZL1, MPZL2, MPZL3, MPZ and JAML, genes are reduced as compared to a comparable cell not containing the inactivating mutations.

In certain aspects, the cell is a haploid cell.

In certain aspects, the cell is a diploid cell.

In certain aspects, the cell is a cultured immortalized cell.

In certain aspects, the cell is a mammalian cell.

In certain aspects, the cell is a human cell.

In certain aspects, the cell is a mouse cell.

In certain aspects, the cultured immortalized cell is a HEK293, Cos7, or CHO cell.

In certain aspects, the beta/MPZΔcell further comprising a defined engineered voltage-gated sodium channel, wherein the engineered voltage-gated sodium channel comprises a sodium channel alpha subunit, at least one sodium channel beta subunit.

In certain aspects, the defined engineered sodium channel beta subunit is encoded by SCN1B, SCN2B, SCN3B, or SCN4B.

In certain aspects, the defined engineered voltage-gated sodium channel further comprises a MPZ family protein. In certain aspects, the sodium channel MPZ family protein subunit is encoded by MPZ, MPZL1, MPZL2 or MPZL3.

In certain aspects, the defined engineered voltage-gated sodium channel further comprises a JAML family protein.

High Through-Put Assays

There is great utility in adapting these engineered cell lines to currently available automated patch-clamp systems, whereby electrophysiological profiles can be simultaneously collected on hundreds of cells in parallel, ensuring robust, and reproducible datasets. This approach provides ample electrophysiological profiles for rigorous characterization of α/β voltage-gated sodium channels (VGSC) complexes and corresponding disease mutants, but also provides a platform to assess more efficiently the effects of multiple small molecule drugs on the behavior of defined α/β VGSC complexes.

Automated patch-clamp systems are available from companies such as Fluxion, Sophion and Nanion. These devices enable drug screening programs based on assessing precise and subtle changes in channel opening and closing behavior. Though changes are subtle, it is precisely these subtle changes that is the goal of developing drugs that target VGSC. It is an important goal to change them slightly, rather than simply block them. This level of precision needs precision cell lines in which to conduct drug screens, which are provided by the present methods.

In certain aspects, provided herein is method of screening effectiveness of a drug targeting a voltage-gated sodium channel (VGSC) disease or disorder. In certain aspects, the beta/MPZΔ cells as well as other cells lacking beta-subunit and MPZ family genes are used for screening new drugs for effects on voltage gated sodium channel function. Cells are transfected to express individual sodium channel alpha subunits and then also transfected to express individual beta-subunit and MPZ family proteins to make voltage-gated sodium channels of a defined composition. High-throughput patch-clamping methods, that work in arrayed format such as a 96 well plate, are then used to measure gating properties of cells in the presence of different drugs. The gating properties are then compared to find drugs that affect them and to determine if those effects are specific to defined sodium channel compositions.

In certain aspect, provided herein is a method of screening effectiveness of a drug targeting a voltage-gated sodium channel (VGSC) disease or disorder, comprising:
(a) culturing a beta/MPZ family-subunit-eliminated (beta/MPZΔ) engineered-cell comprising a cell with functionally inactivated SCN1B, SCN2B, SCN3B, SON4B, MPZL1, MPZL2, MPZL3, MPZ and JAML genes, and comprising a defined engineered voltage-gated sodium channel, wherein the engineered voltage-gated sodium channel comprises a sodium channel alpha subunit, at least one sodium channel beta subunit;
(b) introducing a target drug to the cells;
(c) applying a high-throughput patch-clamping to the cells to measure gating properties of the cells in the presence of the drugs as compared to control cells; and
(d) comparing the gating properties of the beta/MPZΔ cell the gating properties of the controls cell.

In certain aspects, the defined engineered sodium channel beta subunit is encoded by SCN1B, SCN2B, SCN3B, or SCN4B.

In certain aspects, the defined engineered voltage-gated sodium channel further comprises a MPZ family protein. In certain aspects, the sodium channel MPZ family protein subunit is encoded by MPZ, MPZL1, MPZL2 or MPZL3.

In certain aspects, the defined engineered voltage-gated sodium channel further comprises a JAML family protein.

In certain aspect, the VGSC disease or disorder is Long QT syndrome, Brugada syndrome, Atrial standstill, Atrial fibrillation, Sudden infant death syndrome, Sick sinus syndrome, Dilated cardiomyopathy, Generalized epilepsy, Dravet syndrome, Migraine, Autism, Ataxia, Neonatal-infantile seizures, Familial primary erythromelalgia, Hyperkalemic periodic paralysis, Congenital myotonia, or a pain disorder.

### Diseases and Disorders of Interest

This invention provides a way to analyze voltage-gated sodium channels (VGSC) and enables drug discovery programs that require a defined channel composition so that drugs can be tuned to precise targets. VGSCs have several isoforms that collectively inhabit a number of tissues. Their dysfunction can cause both inherited and acquired maladies including cardiac arrythmias, unregulated pain, and epilepsy.

In certain aspects, exemplary VGSC-involved diseases include Long QT syndrome, Brugada syndrome, Atrial standstill, Atrial fibrillation, Sudden infant death syndrome, Sick sinus syndrome, Dilated cardiomyopathy, Generalized epilepsy, Dravet syndrome, Migraine, Autism, Ataxia, Neonatal-infantile seizures, Familial primary erythromelalgia, Hyperkalemic periodic paralysis, Congenital myotonia, and Pain disorders.

siRNA Testing for the Transitory Effect of Knocking Out Gene Function

The goal of the present work was to knock out an entire family of proteins related to the beta subunits of voltage-gated sodium channels to create a ‘tabula rasa’ cell line in which sodium channels of defined composition can be expressed, studied, and used for high-throughput drug screening. Before this work was done, it was not known if such an approach would reveal new and interesting information. Successful results were demonstrated using the BeHAPe cell line.

Crispr/Cas9 technology using guide RNAs and Cas9 was used to put double stranded DNA breaks within the coding region of the SCN1B, SCN2B, SCN3B, SCN4B, MPZL1, MPZL2, MPZL3, MPZ and JAML, genes. Both long-read and short-read DNA sequencing methods, which are commonly known in the art, were used to verify the complete absence of a wildtype allele for all of these genes.

### Definitions

The term “nucleic acid” refers to deoxyribonucleotides or ribonucleotides and polymers thereof in either single- or double-stranded form, composed of monomers (nucleotides) containing a sugar, a phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material, while ribonucleic acid (RNA) is involved in the transfer of information contained within DNA into proteins. The term “nucleotide sequence” refers to a polymer of DNA or RNA that can be single-or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The terms “protein,” “peptide” and “polypeptide” are used interchangeably herein. The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. The present invention provides cells that contain nucleic acids that encode variant SCN1B, SCN2B, SCN3B, SCN4B, MPZL1, MPZL2, MPZL3, MPZ and JAML genes.

“Recombinant DNA molecule” is a combination of DNA sequences that are joined together using recombinant DNA technology and procedures used to join together DNA sequences.

“Wild-type” refers to the normal gene, or organism found in nature without any known mutation.

“Genome” refers to the complete genetic material of an organism.

“Expression” refers to the transcription and/or translation in a cell of an endogenous gene, transgene, as well as the transcription and stable accumulation of sense (mRNA) or functional RNA. Expression also refers to the production of protein.

By “variant” polypeptide or protein is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art. Thus, the polypeptides of the invention may be altered in various ways including amino acid substitutions, deletions, truncations, and insertions. The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.”

“Transformed,” “transgenic,” and “recombinant” refer to a host cell or organism into which a heterologous nucleic acid molecule has been introduced. The nucleic acid molecule can be stably integrated into the genome generally known in the art. For example, “transformed,” “transformant,” and “transgenic” cells have been through the transformation process and contain a foreign gene integrated into their chromosome. The term “untransformed” refers to normal cells that have not been through the transformation process.

The term “nucleic acid” refers to deoxyribonucleic acid (DNA) or ribonucleic acid (RNA) and polymers thereof in either single-or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

The terms “polynucleotide”, “nucleic acid” and “nucleic acid fragment” are used interchangeably herein. These terms encompass nucleotides connected by phosphodiester linkages. A “polynucleotide” may be a ribonucleic acid (RNA) or deoxyribonucleic acid (DNA) polymer that is single-or double-stranded, that optionally contains synthetic, non-natural or altered nucleotide bases. A polynucleotide in the form of a polymer of DNA may comprise one or more segments of cDNA, genomic DNA, synthetic DNA, or mixtures thereof. Nucleotide bases are indicated herein by a single letter code: adenine (A), guanine (G), thymine (T), cytosine (C), inosine (I) and uracil (U). Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated.

Specifically, degenerate codon substitutions may be achieved by generating sequences in which the third position of one or more selected (or all) codons is substituted with mixed-base and/or deoxyinosine residues.

A “nucleotide sequence” is a polymer of DNA or RNA that can be single-stranded or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

“Identity,” as known in the art, is a relationship between two or more polypeptide sequences or two or more polynucleotide sequences, as determined by comparing the sequences. The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to a reference sequence over a specified comparison window. In the art, “identity” also means the degree of sequence relatedness between polypeptide or polynucleotide sequences, as the case may be, as determined by the match between strings of such sequences. “Identity” and “similarity” can be readily calculated by known methods. Certain methods to determine identity are designed to give the largest match between the sequences tested. Methods to determine identity and similarity are codified in publicly available computer programs. Preferred computer program methods to determine identity and similarity between two sequences include, but are not limited to, the GCG program package, BLASTP, BLASTN, and FASTA. The well-known Smith Waterman algorithm may also be used to determine identity.

Nucleic acid molecules encoding amino acid sequence variants of a SCN1B, SCN2B, SCN3B, SCN4B, MPZL1, MPZL2, MPZL3, MPZ and JAML gene are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the target protein.

The invention will now be illustrated by the following non-limiting Examples.

### EXAMPLE 1

### Beta-Subunit-Eliminated eHAP Expression (BeHAPe) Cells Reveal New Properties of the Cardiac Voltage-Gated Sodium Channel

### Abstract

Voltage-gated sodium (Na_{V}) channels drive the upstroke of the action potential and are comprised of a pore-forming α-subunit and regulatory β-subunits. The β-subunits modulate the gating, trafficking, and pharmacology of the-subunit. These functions are routinely assessed by ectopic expression in heterologous cells. However, currently available expression systems may mask the full range of these effects since they contain endogenous β-subunits. To better reveal β-subunit functions, a human cell line devoid of endogenous Na_{V} β-subunits and their immediate phylogenetic relatives was engineered. This new cell line, β-subunit-eliminated eHAP expression cells (BeHAPe), are haploid eHAP cells with inactivating mutations in SCN1B, SCN2B, SCN3B, SCN4B, MPZL1, MPZL2, MPZL3, MPZ and JAML.

Using diploid BeHAPe cells, the effect was examined of β-subunits on the gating properties of the cardiac Na_{V} α-subunit, Na_{V}1.5. These studies revealed that each β-subunit and MPZ set Na_{V}1.5 to unique gating parameters. Furthermore, multiplexing β1 and β2 with Na_{V}1.5generated a sodium channel with hybrid properties, distinct from the effects of the individual subunits. In contrast, in the parental eHAP cell line Na_{V}1.5 gating was unaffected by β1. Thus, BeHAPe cells revealed an expanded ability of β-subunits to regulate Nav1.5 activity and this approach can be used to improve the characterization of other α/β Nav complexes.

### Introduction

Voltage-gated sodium (Na_{V}) channels drive the upstroke of the action potential in excitable cells, thereby generating the electric signals that underlie behavior, sensation, muscle contraction and mobility. Na_{V} channels are composed of an α-subunit and one or two β-subunits. There are ten α-subunit isoforms. The α-subunits are large (˜250 kDa) proteins with 24 transmembrane segments that are arranged in four domains (DI-DIV) to form a central sodium selective and conducting pore. There are also five Na_{V} β-subunit isoforms. The Na_{V} β-subunits, β1-4, are single-pass membrane proteins, with an extracellular immunoglobulin (lg)-like domain. These β-subunits are encoded by the genes SCN1B, SCN2B, SCN3B, and SNC4B. A fifth Na_{V} β-subunit, B1b, generated from a splice variant of SCN1B, is expressed as a secreted protein containing the Ig-like domain. The functions of β-subunits include trafficking and retention of the α-subunit at the plasma membrane, altering the pharmacology of the α-subunit, and altering the voltage-dependent gating properties of the α-subunit. These functions are crucial for proper electrical conduction, as even relatively small changes in the gating equilibria and rates between closed, open, and inactivated states can cause electrical dysfunction.

The predominant Na_{V} α-subunit in the heart is Na_{V}1.5. Na_{V}1.5 forms complexes with β1-β4. Na_{V}1.5 and β-subunits are important in cardiac physiology. Mutations in Na_{V}1.5 or the β-subunits are associated with cardiac arrythmias and disorders such as Brugada Syndrome (BrS) and Long QT syndromes (LQT). Furthermore, mice exhibit cardiac arrhythmia when Na_{V}1.5 or β1-β3 are deleted (the cardiac phenotype of β4 knockout mouse has not been reported). Despite the importance of Na_{V}1.5 and β-subunits, precisely and systematically defining the effect of β-subunits and their diseases variants, on the electrophysiology of Na_{V}1.5 has been challenging. This is, in part, because of the limitations of currently available expression systems.

The electrophysiological properties of Na_{V} channels are routinely determined in heterologous systems, such as HEK293T, CHO and COS cells. These systems lack endogenous voltage-gated ion channels, allowing for the current generated only by ectopically expressed NaV α-subunit to be measured. However, they express endogenous β-subunits, which could alter α-subunit properties and interfere with the response to ectopically expressed β-subunits. To partially circumvent this issue Xenopus laevis oocytes are sometimes used as they have minimal endogenous β-subunit expression. But as X. laevis oocytes require a low incubation temperature (18° C.) they are not ideal for the study of disease variants. In addition to interference from endogenous canonical β-subunits, the presence of phylogenetic relatives of β-subunits may also modify Na_{V} channels. The β-subunits belong to a sub-family of proteins (β/MPZ) that includes MPZ, MPZL1, MPZL2, MPZL3 and JAML. Although not implicated in directly regulating Na_{V} channels, these proteins may regulate the channel in cultured cells if they share biophysical properties with β-subunits. Indeed, the sequences of β1 and β3 are as similar to the MPZ subfamily as they are to β2 and β4. Thus, an ideal expression system for studying Na_{V} β-subunit isoforms, and their disease associated alleles, should lack an endogenous β/MPZ family.

In this study CRISPR-Cas9 gene-editing was used to generate β-subunit-eliminated eHAP expression (BeHAPe) cells, a cell line devoid of the β/MPZ family. This cell line allows for β-subunits to be expressed as the sole-subunit family member without interference from endogenous β-subunits. These cells were used to assess the effects of β-subunits on the gating properties on the predominant cardiac α-subunit Na_{V}1.5. The BeHAPe cells exhibited enhanced sensitivity to β1 compared to parental eHAP cells. The data show that co-expressed β-subunits generated a repertoire of Na_{V}1.5 channels with unique gating properties. These findings demonstrate that this approach improves the characterization of the electrophysiological properties of α/β Na_{V} complexes.

### Methods

### Molecular Biology Reagents

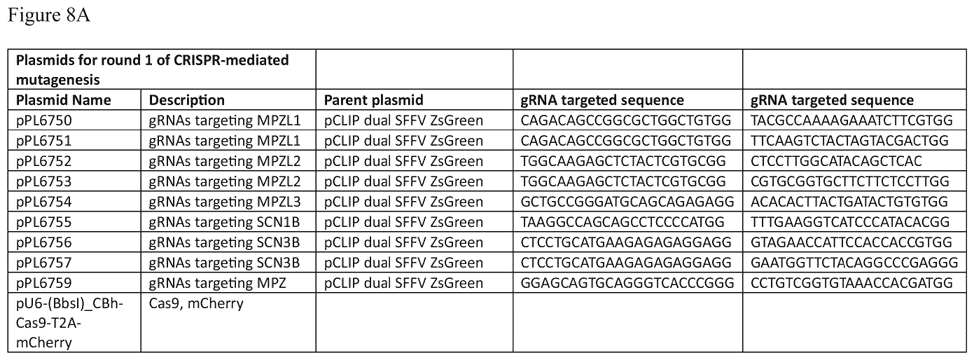

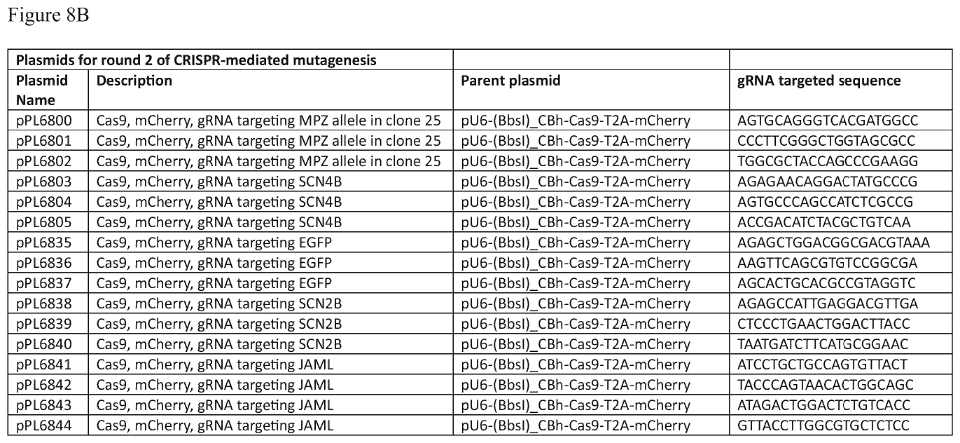

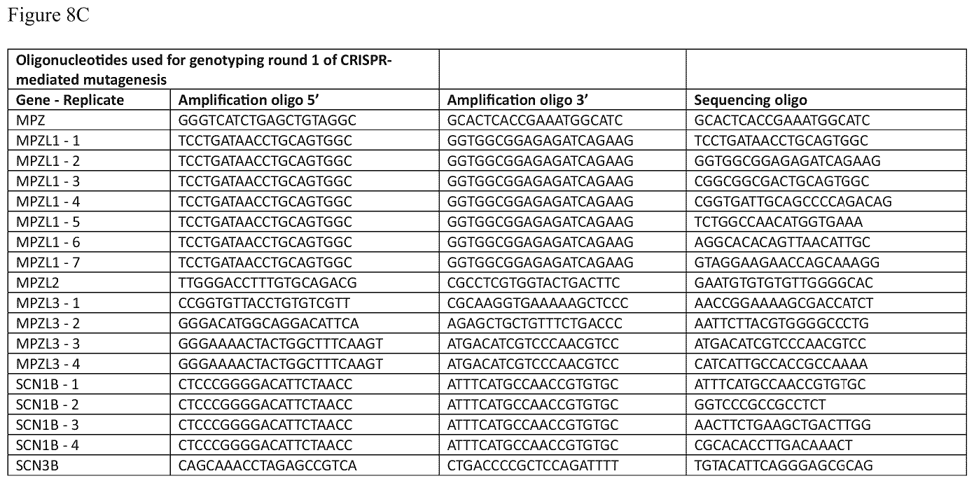

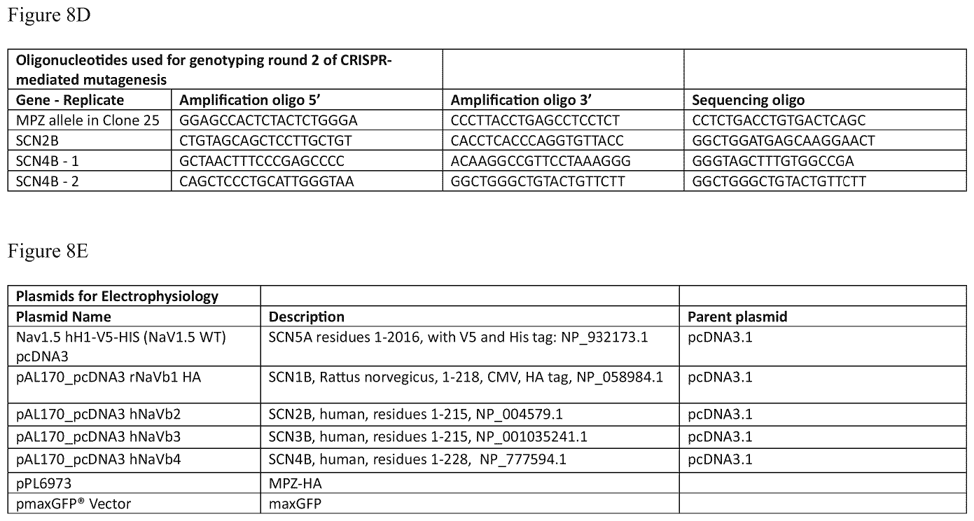

Plasmids and oligonucleotides used in this study are described in FIGS. 8A-8E. All enzymes targeting DNA and RNA were obtained from New England Biolabs (Ipswitch, MA).

### Cell Culture

Human haploid eHAP cells (Cat #C669, Horizon Discovery, Cambridge MA) were cultured in Iscove's Modified Dulbecco's Medium (IMDM: Gibco, Billings, MT) supplemented with 10% FCS. Cells were passaged every 48-72 h. As noted previously, eHAP cells can diploidize and make aneuploid populations. Thus, eHAP cells and their derivates were enriched for haploid cells using flow-actuated cell sorting (FACS) using size (FSC: forward scatter; SSC: side scatter) as the sorting parameter. Haploid-enriched populations of eHAP cells were used for gene engineering, whereas diploid cells were used for electrophysiology experiments. For transfection, Lipofectamine 3000 (Invitrogen/Thermofisher, Waltham, MA) was used in a reverse-transfection scheme using manufacturer's instructions for making DNA-Lipofectamine particles. Some culture conditions required conditioned media, which was made as follows: BeHAPe cells were seeded onto a 100 mm dish, allowed to become confluent, after which they were grown for an additional week. The media was then harvested and passed through a 0.22 μm filter prior to storage at 4° C. until used.

### Generation of BeHAPe Cells

A Flp Recombination Target site (FRT) was first integrated into eHAP cells. eHAP cells were transduced with lentivirus carrying the pQCXIP FRT GFP-Neo^{R} (pPL6490) vector. pPL6490 encodes an FRT site, GFP and neomycin-resistance. pPL6490 was derived from pQCXIP, a bicistronic retroviral expression vector that originally conferred puromycin-resistance. pPL6490 was packaged into lentivirus in HEK293 cells using the previously described lentivirus packaging system. Transduced eHAP cells were selected by isolating neomycin-resistant colonies that express GFP. From these clones eHAP-FRT was identified. The FRT integration site in eHAP-FRT was defined by isolating the integrated plasmid DNA and the genomic DNA flanking the integration site. This was achieved by first digesting genomic DNA with the restriction enzymes HindIII-HF, EcoRV-HF, HpaI, ApaI, BamHI-HF, XhoI, StuI, as well as RNAaseA and Klenow fragment in the presence of 1 mM dNTPs. Digested genomic DNA was ligated with T4 DNA ligase and electroporated into SURE bacterial cells (Agilent, Santa Clara, CA). Plasmids from ampicillin-resistant colonies were Sanger sequenced to define the FRT insertion described.

To disrupt genes encoding the B/MPZ family, two rounds of CRISPR-Cas9 gene-editing were performed on eHAP-FRT cells. Multiple gRNA sequences that targeted the N-terminal signal sequences or the exofacial Ig-like domains were used per gene. In Round 1, eHAP-FRT cells were transiently transfected with pU6-(BbsI)_CBh-Cas9-T2A-mCherry encoding S. pyogenes Cas9 and mCherry, and nine plasmids derived from pCLIP dual SFFV ZsGreen (Transomics, Huntville AL) that encoded gRNAs against MPZ, MPZL1, MPZL3, SCN1B, SCN3B. After 48 h, ZsGreen and mCherry double-positive cells were isolated by FACS as described below, and serially diluted into 100 mm plates and allowed to form colonies. Colonies were isolated using trypsin-soaked discs as previously described and assessed for ploidy using propidium iodide staining. Haploid populations were genotyped as described below. One clone (clone 25) had frame-shifting indel mutations in all targeted genes, although MPZ had an in-frame deletion. Clone 25 cells were FACS-sorted for haploid cells and then subjected to another round of CRISPR/Cas9 mediated mutagenesis targeting SCN2B, SCN4B, JAML, MPZ and EGFP at the FRT locus. Here, gRNA encoding sequences were cloned into the BbsI site downstream of the U6 promoter of pU6-(BbsI)_CBh-Cas9-T2A-mCherry to generate 16different plasmids. Clone 25 cells were transiently transfected with these 16 plasmids, and 48 h after transfection, cells positive for mCherry were isolated by FACS, serially diluted, and allowed to form colonies after plating. Clones were isolated on cloning discs, and after expansion were assessed for ploidy using propidium iodide staining and genotyped. One clone, BeHAPe cells, had frame-shifting indel mutations in all targeted genes, except for MPZ and SCN2B, which acquired a larger in-frame deletion. BeHAPe cells were FACS-sorted for haploid cells before storage. BeHAPe cells are available upon request from Robert Piper or Chris Ahern, University of Iowa.

Genotyping of eHAP-Derived Cells

Genomic DNA was harvested using proteinase K and phenol-chloroform extraction. The CRISPR-targeted genetic loci were PCR amplified from genomic DNA using NEBNext High-Fidelity 2X PCR Master Mix and the primer pairs in FIGS. 8A-8E. PCR products were purified, and Sanger sequenced using the primers also listed in FIGS. 8A-8E. Whole genome sequencing was performed using an Oxford Nanopore MinION equipped with a version 10.41 flow cell. Reads were mapped using miniMAP2 using the default parameter for Oxford Nanopore data.

### Ploidy Analysis and FACS

The ploidy of eHAP cells was determined by propidium iodide staining and analyzed on a Becton Dickinson LSR II flow cytometer. Cells were trypsinized, washed twice with PBS, lysed and stained using Nicoletti buffer (0.1% sodium citrate, 0.1% Triton X-100, 0.5 unit/mL RNase A, 20 units/mL RNase T1, 50 μg/mL propidium iodide). Haploid eHAP cells were used for reference. Ploidy was also assessed by Hoescht 33342 staining. Hoescht 33342 (5 ug/mL) was applied to live eHAP cells for 10 min, cells were then trypsinised and fluorescence intensity measured on a Becton Dickinson Aria II. Cells showed strong correlation between cell size and Hoescht intensity allowing the former parameter to be used to enrich haploid cells via cell sorting.

For cell sorting experiments, a Becton Dickinson Aria II equipped with a 130 μM diameter nozzle was used. Haploid or diploid cells were enriched by gating based on FSC and SSC. Reference populations were used to define the gates. Cells transiently expressing mCherry and/or ZsGreen were sorted by using gates that captured the brightest 0.3% of cells.

### Preparation of Cells for Electrophysiology Experiments

Electrophysiology experiments were performed on diploid BeHAPe cells using ‘reverse’ transient transfection of the plasmids listed in FIGS. 8A-8E. All constructs were grown in DH5α cells (NEB #2987H), prepared using PureLink HiPrep plasmid preparation kits (Invitrogen/Thermofisher, Waltham, MA), followed by sequencing of the reading frame and promoter. All constructs expressed their respective open-reading frame via the CMV promoter. Cells were transfected with plasmids expressing Na_{V}1.5-V5, pmaxGFP expressing Pontellina plumata GFP (Amaxa Biosciences, Cologne, Germany), and either empty vector (pcDNA3.1) or plasmid expressing a β/MPZ family member using a ratio of 2:1:2, respectively. 24 h after transfection, cells were dissociated from tissue culture dish using Versene (Gibco, Billings, MT), washed and resuspended in conditioned IMDM with 10% FCS. Cells were then immediately passed through a 10 μM nylon net filter (Milipore) and dispersed further by gentle pippetting before plating in 35 mm corning cell culture dishes (SKU: CLS430165, Corning, Tewksbury, MA). 12-24 h after reseeding cells, electrophysiology experiments were performed.

### Whole-Cell Voltage Patch Clamp

Ionic currents through Na_{V}1.5 (SCN5A) channels expressed in BeHAPe cells were recorded using whole-cell patch on Axon Axopatch 200B amplifiers (Molecular Devices, San Jose, CA). Data were collected and analyzed with pClamp11/Clampfit11 (Molecular Devices, San Jose, CA) and Origin software (OriginLab, Northhampton, MA). Glass microelectrodes had resistances of 1.5-2 M Ω. Internal solution consisted of 105 mM CsF, 33 mM NaCl, 10 mM HEPES, 10 mM EDTA, pH-adjusted to 7.3 with CsOH. External solution contained 150 mM NaCl, 2 mM KCl, 1.5 mM CaCl₂, 1 mM MgCl₂, 10 mM HEPES, pH-adjusted to 7.4 with NaOH. Included recordings had currents between 1 nA and 12 nA, access resistance of <6 MΩ, and compensated series resistance of >90%. All cells were recorded 36-48 h post-transfection. Data from at least 3 separate transfections constituted each dataset. Cells of similar size were analyzed by recording only from cells with whole-cell capacitance between 0.5-1.0 pF. Data were sampled at 20 kHz and filtered at 5 kHz. Leak currents were subtracted with the p/8 protocol, except in the case of the β-subunit only control conditions wherein the minute leak current was not subtracted to ensure integrity of control measurements. The Conductance-Voltage relationship (GV) curves were determined by dividing the measured current amplitude at a given test voltage by the driving force. Each GV and Steady State Inactivation (SSI) was individually fit to a Boltzmann function confined between 0-1 and averaged to produce experimental values. For rates of inactivation, all data were P/8-subtracted, and currents were fit to a single exponential with the function y=A+A₀ exp^{-t/t}, with the bounds of the fit currents set manually to include the inactivating portion of the trace. Multiple authors conducted blinded exponential fits ensure fit integrity.

### Statistical Analysis

Statistical analyses were conducted using GraphPad Prism 9. One-way ANOVA with Dunnet correction for multiple comparisons was performed for pairwise comparisons of multiple groups to the control sample and data are presented as mean±SD. Paired comparisons were also evaluated by T-test and presented as mean±SD.

### Results

### Generation of BeHAPe Cells

To better reveal how β-subunits modulate the gating properties of α-subunits, a cell line was generated devoid of the β-subunit family (SCN1B, SCN2B, SCN3B, SCN4B) and their phylogenetic relatives (MPZ, MPZL1, MPZL2, MPZL3 and JAML). These genes were deleted from human haploid (eHAP) cells. eHAP cells are fibroblast like cells that do not produce endogenous voltage-gated sodium currents. These cells are also haploid, simplifying mutagenesis and genotyping. Previous RNAseq experiments (Accession: SRX655513, SRX65551) show that this cell line has low to moderate expression of β/MPZ family members. No expression of SCN2B or JAML was detected, whereas SCN1B, SCN3B, SCN4B, MPZ, MPZL1, MPZL2, and MPZL3 had FPKM values of 0.70, 0.20, 0.016, 2.15, 3.40, 0.01, and 0.30, respectively, comparable to those in HeLa and HEK293 cells (SRR1567907, SRR5011299).

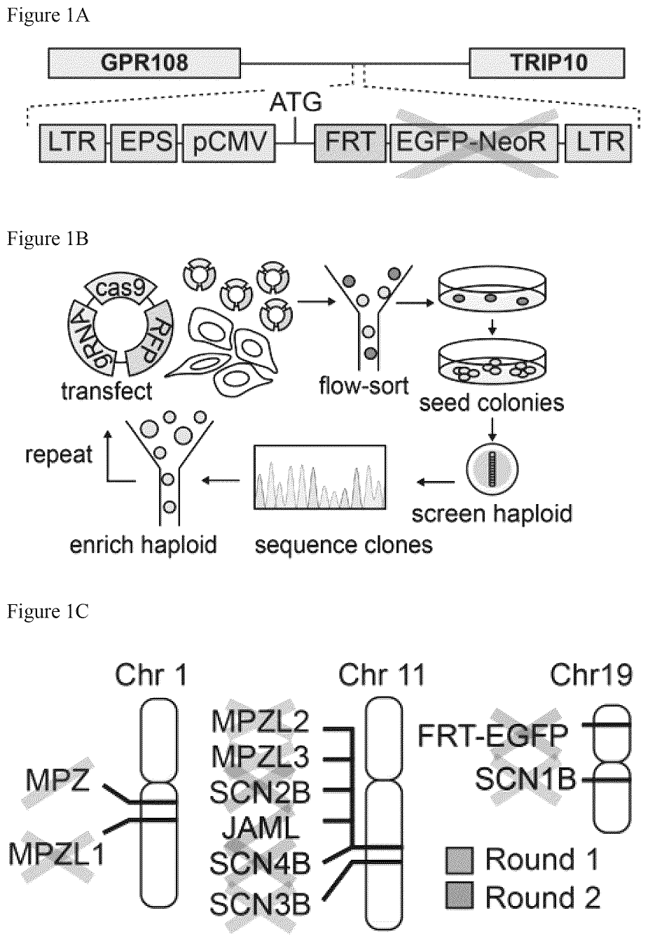

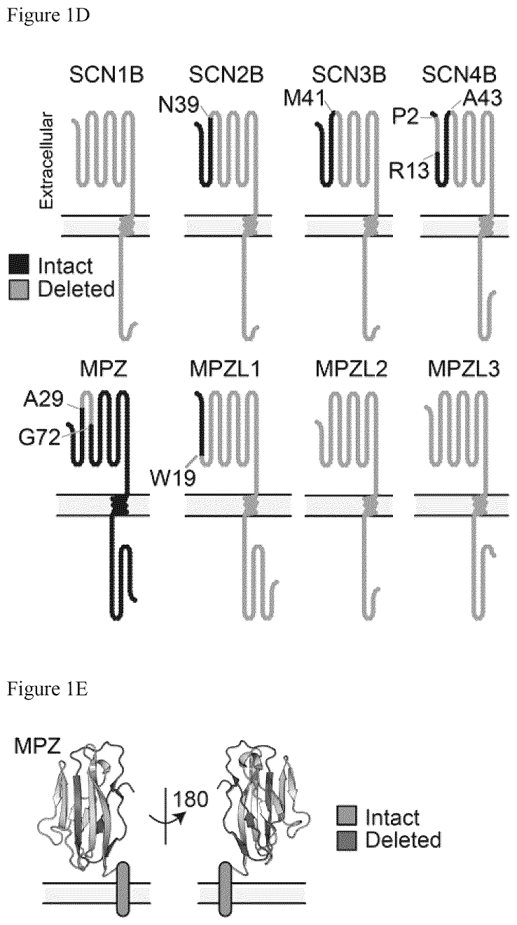

First, an FRT site was integrated into eHAP cells to allow for site-directed recombination in future studies (FIG. 1A). The FRT site was encoded in the pQCXIP FRT GFP-Neo vector, which was transduced into eHAP cells by retrovirus. The integration site was mapped to chromosome 19, in between GPR108 and TRIP10 genes. The introduced locus expressed EGFP fused to neomycin resistance gene (eGFP-Neo^{R}) under a CMV promoter (pCMV). An FRT site was placed between the start codon and eGFP. Later, in the second round of CRISPR-Cas9 deletions, eGFP-Neo^{R}, was deleted leaving the CMV promoter and FRT site available for optional use.

Next, the β/MPZ family in eHAP-FRT cells was disrupted in two rounds of CRISPR-Cas9 gene editing (FIG. 1B). For these experiments, Cas9 and gRNA encoding plasmids, co-expressing fluorescent reporter proteins, were transiently transfected into cells. To edit nine genes in two rounds of CRISPR-Cas9, multiple gRNA per gene were introduced and targeted multiple genes in each round. To improve the efficiency of gene edits, haploid cells were enriched for prior to gene editing, which was necessary as eHAP cells diplodize. Fluorescent transfected cells were enriched by FACS given that transfection efficiency was roughly 30%. As eHAP cells are haploid, the genomic sequence of all the targeted β/MPZ genes could be determined by Sanger sequencing. Knockouts were identified by selecting cells with indel mutations that frame-shifted the protein coding sequence or removed the start codon. Oxford nanopore was later used to confirm long insertions/more complicated rearrangements.

After the first round of mutagenesis, a cell line (clone 25) was generated that contained frame-shifting indel mutations in MPZL1, MPZL3, SCN3B, an in-frame deletion that removed the start codon of SCN1B, and an in-frame deletion of MPZ (FIG. 1C). Clone 25 was subject to a second round of mutagenesis, and this led to the generation of BeHAPe cells. BeHAPe cells contain additional frame-shifting indel mutations in SCN4B and EGFP, an in-frame deletion of SCN2B and a larger in-frame deletion of MPZ. Exon 1 and 2 of JAML were also targeted in BeHAPe cells but could not be PCR amplified and sequenced, indicating there was likely a large disruption in the JAML locus.

The impact of the gene disruptions at the protein level are shown in FIG. 1D. The gene-edits completely disrupted the protein sequences of SCN1B, MPZL2, MPZL3, while the protein sequences of SCN3B, SCN4B and MPZL1 encode only a truncated N terminus. While the deletion in MPZ left the codons for the signal sequence (MI-A29) intact, it removed 130-E71, which encodes for a central β-strands in the Ig domain (FIG. 1E). This deletion should render the Ig domain of MPZ misfolded and possibly subjected to ER associated degradation, as is the fate of MPZ mutants associated with Charot-Marie-Tooth disease. Similarly, a portion of SCN2B was deleted, disrupting the Ig domain.

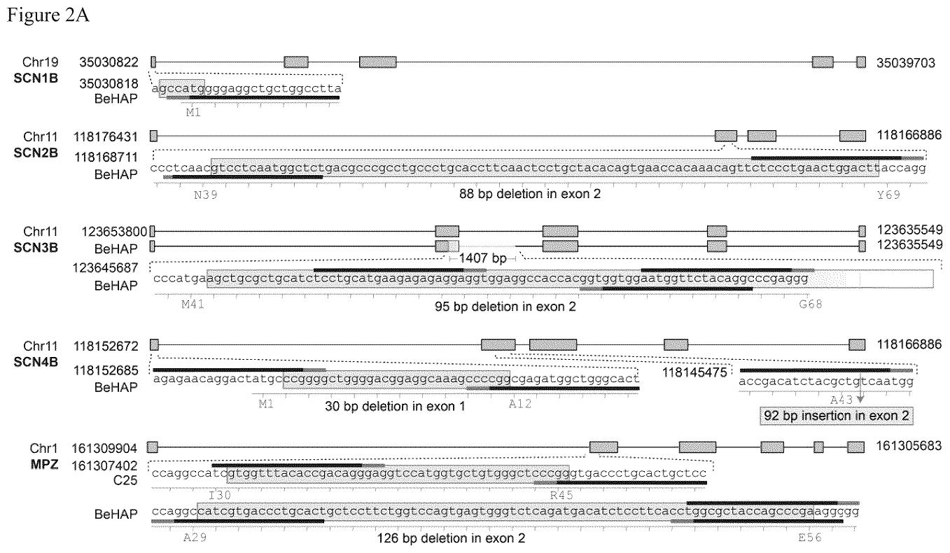

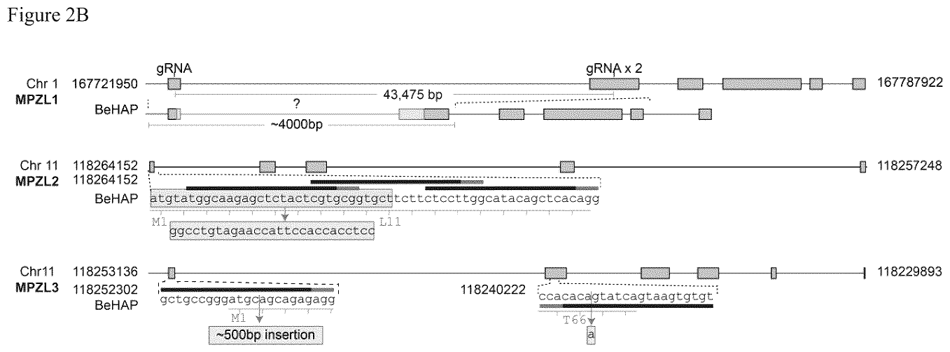

The edited genomic loci in BeHAP cells are illustrated in FIGS. 2A-2B. In SCN1B, exon 1 had a 6 bp deletion that included the start codon. In SCN2B, exon 2 had a frame-shifting 88 bp deletion. In SCN3B, 1407 bp were deleted including a frame-shifting 95 bp from 3′ end of exon 2. In SCN4B, exon 1 had an in-frame 30 bp deletion and exon 2 had a frame-shifting 92 bp deletion. In, MPZ exon 2 had an in-frame 126 bp deletion. In MPZL1, a large 43,475 bp region that included portions of exon 1 and 2 was deleted and replaced with ˜4000 bp of repetitive sequence. In MPZL2, a frame-shifting 32 bp from exon 1, which included the start codon, was replaced with 27 bp which kept the protein coding sequence out of frame. In MPZL3, ˜500 bp of repetitive sequences was inserted just after the encoded initiating Met. There was also a single bp insertion in exon 2, causing a frameshift mutation.

The gene disruptions at the DNA level are shown in FIG. 2A-2B. In SCN1B, exon 1 had a 6 bp deletion that included the start codon. In SCN2B, exon 2 had an 88 bp deletion. For SCN3B, 1407 bp were deleted including 95 bp from 3′ end of exon 2. For SCN4B, exon 1 had a 30 bp deletion and exon 2 had a 92 bp deletion. For, MPZ exon 2 had a 126 bp deletion. For the MPZL1 locus, a large 43,475 bp region that included portions of exon 1 and 2 was deleted and replaced with ˜4000 bp of repetitive sequence. For MPZL2, 32 bp from exon 1, which included the start codon, was replaced with 27 bp. For MPZL3, ˜500 bp of repetitive sequences was inserted just after the encoded initiating Met. There was also a single bp insertion in exon 2, causing a frameshift mutation. There was also a single bp insertion in exon 2, causing a frameshift mutation. In JAML, exon 2 had a frame-shifting ˜2888 bp insertion. Genotyping of the disrupted JAML locus could not be accomplished with Sanger sequencing, but was accomplished using genomic sequencing using long read technology.

Surprisingly, only in-frame deletions of MPZ were recovered. In the first round of mutagenesis only a single clone (Clone 25) with an edited locus was recovered, and its locus had an in-frame deletion (FIG. 2A-2B). In the second round of mutagenesis several clones with further edited MPZ were recovered, yet all had in-frame edits. This included three clones with in-frame deletions; one clone with a frameshift deletion, which was corrected with a second frameshift deletion that restored the wildtype reading frame; and two clones that had replaced sequences with new sequences that preserved the reading frame. All these deletions left the signal sequence intact, and the largest span of deleted residues was between 130-E71. These data suggest that this protein may be essential in haploid eHAP cells. If so, the essential function it serves must not require the extracellular Ig domain since this domain was disrupted in BeHAPe cells. The major known function of MPZ is in the compaction of myelin, a function irrelevant to eHAP viability. In addition, MPZ knockout mice are viable indicating MPZ is not essential in multiple cell types.

### Patch-Clamping BeHAPe Cells

It was next sought to examine the electrophysiological properties of the cardiac Na_{V} channel Na_{V}1.5 in BeHAPe cells. However, whole cell patch-clamp experiments were initially challenging because BeHAPe cells were small, exhibited a low transfection efficiency, and a plasma membrane that was prone to blebbing, which made it difficult to obtain tight giga-Ohm seals. This plasma membrane morphology was not observed in eHAP cells, which were easier to patch-clamp. These problems were partially mitigated through the following optimization steps.

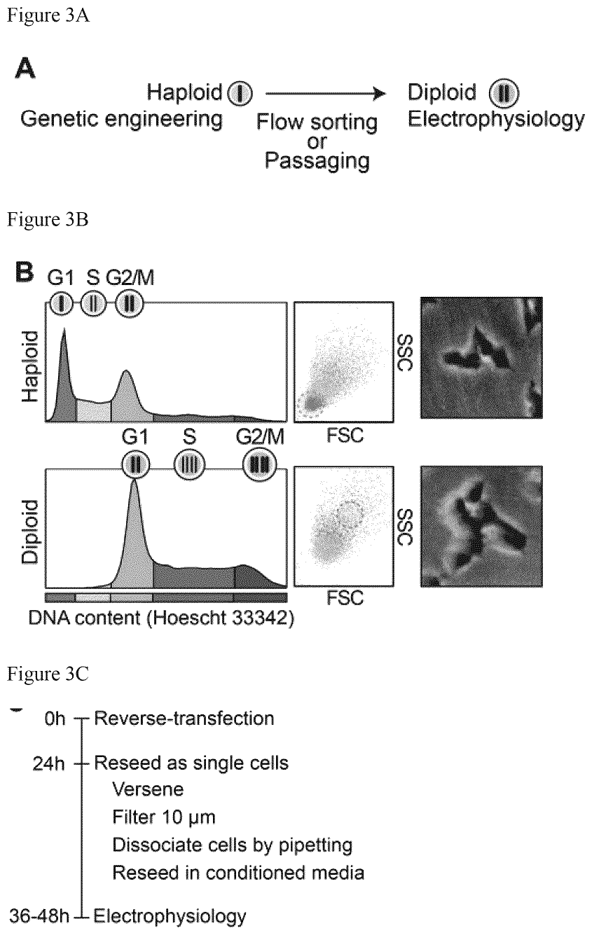

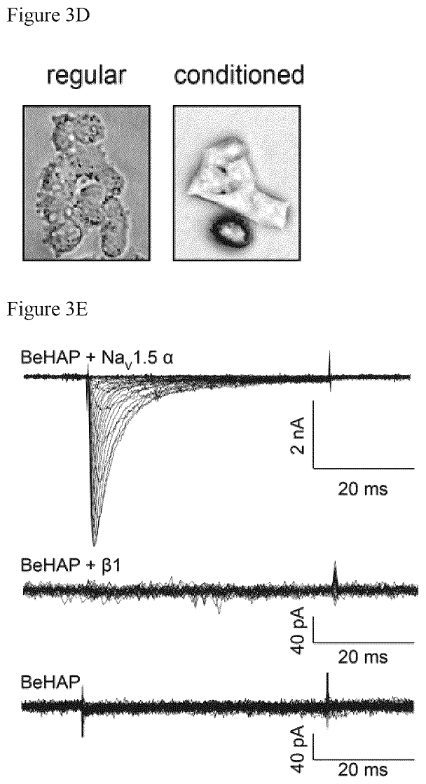

First, while haploid BeHAPe cells (and parental eHAP cells) are small, diploid BeHAPe cells are larger and easier to patch-clamp (FIGS. 3A-3E). A diploid population was obtained by passaging haploid cells, which spontaneously diplodize, and sorting for diploid cells by FACS based on forward (FSC) and side-scatter (SSC) profiles. It was confirmed that the magnitude of FSC and SSC corresponded to haploid and diploid eHAP cells by staining DNA content in live cells using Hoescht 33342 (FIG. 3B). As Hoescht 33342 was toxic to cells, for routine sorting FSC and SSC gates were set using unstained reference populations.

Second, the highest expression of Na_{V}1.5 was achieved, with ˜30% transfection efficiency, in BeHAPe cells when they were ‘reverse’-transfected. Cells were reseeded as single cells 24 hours after transfection and patch-clamped 12-16 hours later, when there was peak expression of a tracer plasmid pmaxGFP (FIG. 3C). As the expression of transgenes were variable, only cells with similar levels of the GFP tracer and current density were analyzed. This selection approach however, precluded the use of peak current density as a proxy for trafficking of the a-subunit to the plasma membrane.

Finally, the plasma membrane of BeHAPe cells was prone to blebbing. The membrane blebbing was not suppressed by re-expression of β/MPZ subunits. Plasma membrane blebbing could, however, be suppressed by growing BeHAPe cells in conditioned media and by avoiding sheer stress (FIG. 3D). To reseed cells as single cells prior to electrophysiology, the amount of pipetting was reduced, and thus shear stress, by passing cells through a 10 μm filter, which is approximately the diameter of a cell, and seeding dissociated cells in conditioned media. During patch-clamp experiments, the cells retained good morphology in external solution for 30 minutes.

These methods allowed whole cell patch-clamp experiments on BeHAPe cells. Like eHAP cells, BeHAPe cells show no voltage dependent currents when transfected with an empty plasmid vector or β-subunit (FIG. 3E). Robust currents are produced only after transfection with the α-subunit Na_{V}1.5. Thus, this system can be used to reliably measure the electrophysiological properties of Na_{V} channels, with the exception of peak current density.

Na_{V}1.5 in eHAP vs BeHAPe Cells

The electrophysiological properties were evaluated of Na_{V}1.5 in BeHAPe cells vs their parental eHAP cell counterparts (FIG. 4). This was done in the presence and absence of β1 co-expression to determine whether eliminating the endogenous β/MPZ genes made a difference in the ability to observe β1-dependent effects on Na_{V}1.5. The data revealed that Na_{V}1.5 alone exhibited similar GV and SSI in BeHAPe and eHAP cells, but the rate of inactivation was slower in BeHAPe cells at −40 mV. In contrast, the properties of Na_{V}1.5+β1, were markedly different in eHAP vs BeHAPe cells. While β1 modulated the GV, SSI, and rate of inactivation of Na_{V}1.5 in BeHAPe cells, in eHAP cells no effect was observed of β1 on any of these properties. Thus, these data demonstrate that eliminating endogenous β/MPZ family in BeHAPe cells sensitized this cell line to β1 effects, and thus is consistent with the proposition that eliminating endogenous β-subunits improves the detection of exogenous β-subunit effects.

### Modulation of Na_{V}1.5 by β/MPZ Proteins in BeHAPe Cells

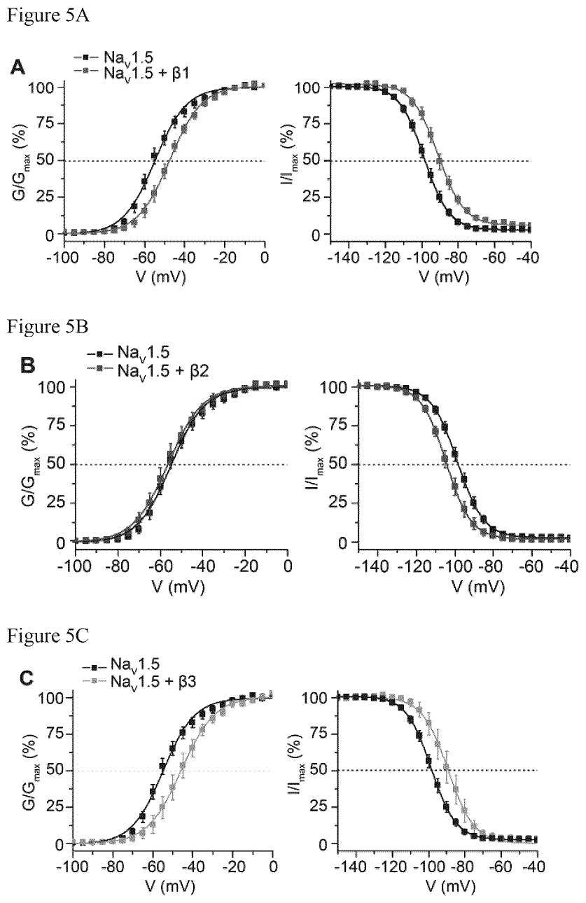

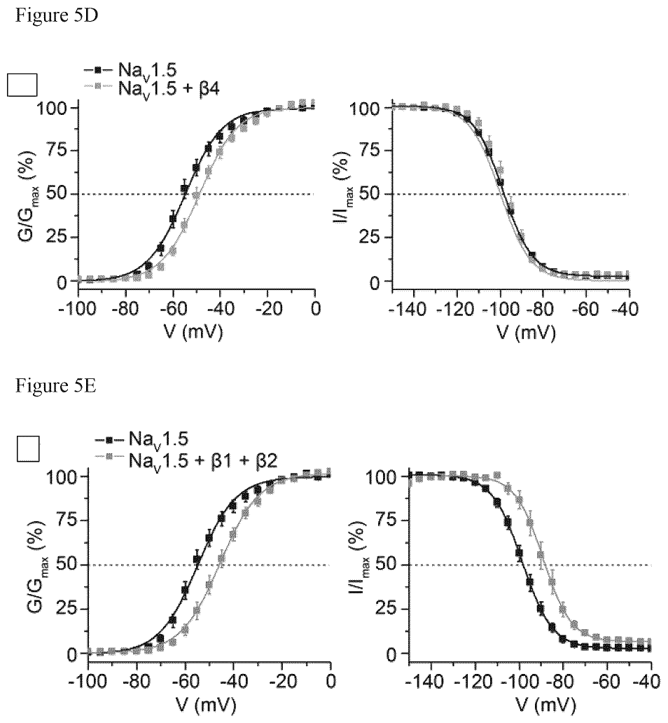

BeHAPe cells were used to examine the effects of β-subunits, 1-4, on the gating of the major cardiac α-subunit Na_{V}1.5. Na_{V}1.5 activity was monitored by whole cell-patch clamp. MPZ was included in the analysis to gauge the capacity of β-subunit phylogenetic relatives to affect Na_{V}1.5 gating. Co-expression of Na_{V}1.5 and β1 shifted the midpoint of the voltage dependence of activation (GV) and steady-state inactivation (SSI) curves to more positive potentials (7.5±0.6 mV for GV, 7.5±0.4 mV for SSI) without altering the slope of these curves (FIGS. 5A-5F and Table 1).

TABLE 1
Gating properties of Na_{V}1.5 in BeHAPe cells

| | GV | | | | SSI | | | | τ inactivation (at V_{1/2}) | | |
|---|---|---|---|---|---|---|---|---|---|---|---|
| | V_{1/2} | ΔV_{1/2} | k | | V_{1/2} | ΔV_{1/2} | k | | τ | Δ τ | |
| Channel | (mV) | (mV) | (Slope) | N | (mV) | (mV) | (Slope) | N | (ms) | (ms) | N |
| eHAP | −49.8 ± 7.1 | — | 8.2 ± 0.4 | 8 | −98.4 ± 7.3 | — | 7.0 ± 0.4 | 6 | 3.0 ± 0.9 | — | 8 |
| Na_{V}1.5 | | | | | | | | | | | |
| eHAP | −53.4 ± 5.5 | 0.2 ± 3.3 | 8.0 ± 0.3 | 8 | −94.6 ± 7.7 | 3.2 ± 3.4 | 7.4 ± 0.3 | 8 | 2.9 ± 0.4 | −0.3 ± 1.0 | 8 |
| Na_{V}1.5 + β1 | | (p = 0.77) | | | | (p = 0.30) | | | | (p = 0.839) | |
| BeHAPe | −53.6 ± 5.6 | — | 7.9 ± 0.4 | 10 | −97.8 ± 3.6 | — | 6.8 ± 0.2 | 9 | 3.2 ± 0.6 | — | 10 |
| Na_{V}1.5 | | | | | | | | | | | |
| BeHAPe | −46.9 ± 4.9 | 6.7 ± 3.2* | 8.0 ± 0.4 | 8 | −89.0 ± 4.9 | 8.8 ± 2.9** | 7.1 ± 0.3 | 7 | 1.5 ± 0.4 | −1.7 ± 1.0** | 8 |
| Na_{V}1.5 + β1 | | (p = 0.020) | | | | (p = 0.0088) | | | | (p = 0.00025) | |
| BeHAPe | −56.1 ± 6.1 | −2.5 ± 3.4 | 8.2 ± 0.5 | 6 | −107.7 ± 6.5 | −9.9 ± 3.1** | 6.4 ± 0.3 | 6 | 2.7 ± 0.7 | −0.5 ± 1.1 | 6 |
| Na_{V}1.5 + β2 | | (p = 0.33) | | | | (p = 0.0019) | | | | (p = 0.30) | |
| BeHAPe | −44.8 ± 4.6 | 8.8 ± 3.1** | 7.4 ± 0.9 | 7 | −88.7 ± 6.3 | 9.1 ± 3.1** | 7.2 ± 0.5 | 6 | 3.7 ± 1.8 | 0.4 ± 1.5 | 6 |
| Na_{V}1.5 + β3 | | (p = 0.0030) | | | | (p = 0.0053) | | | | (p = 0.34) | |
| BeHAPe | −48.4 ± 3.4 | 5.2 ± 3.0* | 8.0 ± 0.3 | 6 | −95.4 ± 4.4 | 2.4 ± 2.8 | 7.0 ± 0.4 | 6 | 3.7 ± 0.9 | 0.5 ± 1.2 | 6 |
| Na_{V}1.5 + β4 | | (p = 0.045) | | | | (p = 0.46) | | | | (p = 0.30) | |
| BeHAPe | −45.7 ± 4.3 | 7.9 ± 3.1** | 8.1 ± 0.4 | 7 | −88.5 ± 5.4 | 9.1 ± 3.0** | 7.2 ± 0.4 | 7 | 3.2 ± 1.1 | 0.0 ± 1.3 | 7 |
| Na_{V}1.5 + | | (p = 0.0073) | | | | (p = 0.0031) | | | | (p = 0.95) | |
| β1 & β2 | | | | | | | | | | | |
| BeHAPe | −44.9 ± 6.5 | 8.7 ± 3.3** | 7.4 ± 0.3 | 7 | −91.5 ± 4.7 | 6.3 ± 2.9* | 7.4 ± 0.3 | 6 | 3.0 ± 0.8 | 0.2 ± 1.2 | 6 |
| Na_{V}1.5 + | | (p = 0.0048) | | | | (p = 0.035) | | | | (p = 0.65) | |
| MPZ | | | | | | | | | | | |
| eHAP | −49.8 ± 7.1 | 3.8 ± 3.5 | 8.2 ± 0.4 | 8 | −98.4 ± 7.3 | −0.6 ± 3.3 | 7.0 ± 0.4 | 6 | 3.0 ± 0.9 | −0.2 ± 1.2 | 8 |
| Na_{V}1.5 | | (p = 0.13) | | | | (p = 0.85) | | | | (p = 0.57) | |
| eHAP | −53.4 ± 5.5 | 0.2 ± 3.3 | 8.0 ± 0.3 | 8 | −94.6 ± 7.7 | 3.2 ± 3.4 | 7.4 ± 0.3 | 8 | 2.9 ± 0.4 | −0.3 ± 1.0 | 8 |
| Na_{V}1.5 + β1 | | (p = 0.80) | | | | (p = 0.44) | | | | (p = 0.21) | |

Gating properties of Na_{V}1.5 in BeHAPe cells. Data are reported as mean ± SD.
*p < 0.05,
**p < 0.01 by one-way ANOVA with Dunnet correction for multiple comparisons. The data in this table were used to generate FIG. 4 and FIGS. 5A-5F.

In contrast, combining Na_{V}1.5 with β2 shifted the midpoint of the SSI curve to more negative potentials (−5.9±0.5 mV) but did not shift the GV curve (FIGS. 5A-5F) or the slope of either curve (Table 1). The effect of β3 on GV and SSI were almost identical to β1, with it shifting the midpoint of the GV and SSI curves more positive (7.4±0.9 m V for GV and 9.4±0.6 m V for SSI) without altering slope (FIGS. 5A-5F and Table 1). β4 also shifted the midpoint of the GV curve to more positive potentials (6.1±0.6 mV shift in GV) but, unlike the other β-subunits did not shift the midpoint of the SSI curve (FIGS. 5A-5F). It was surprising to find substantial effects on Na_{V}1.5 gating by MPZ, which also shifted the midpoint of the GV and the SSI curves to more positive potentials (7.4±0.4 mV for GV and 6.9±0.3 mV for SSI). This effect was similar to those of β1 and β3 (FIGS. 5A-5F and Table 1).

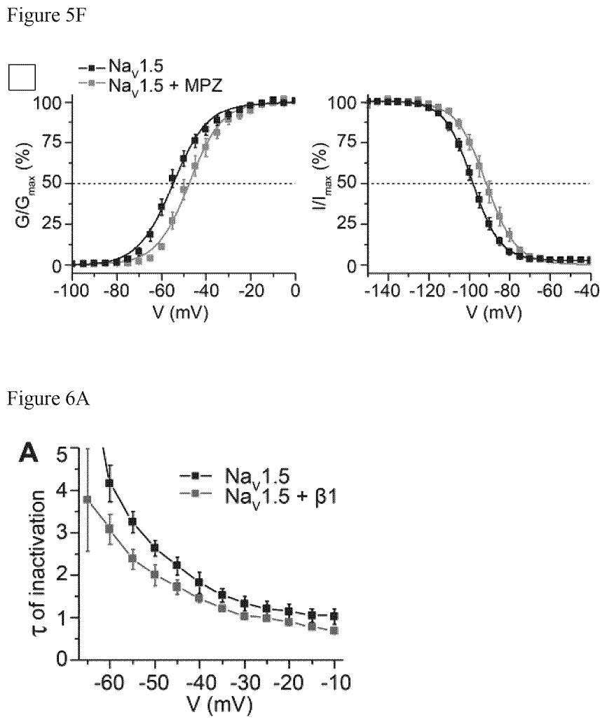

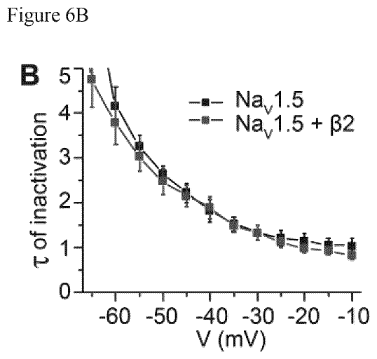

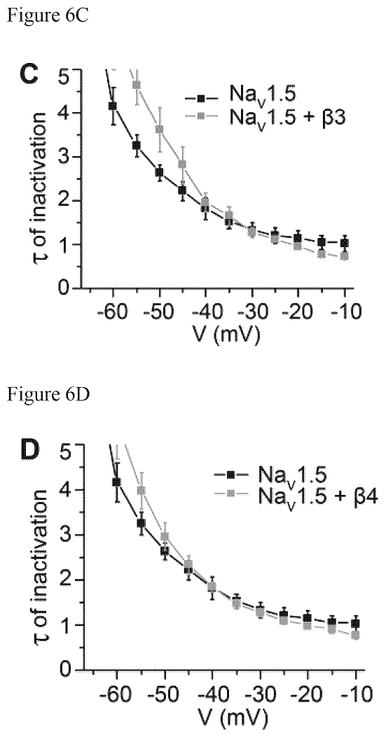

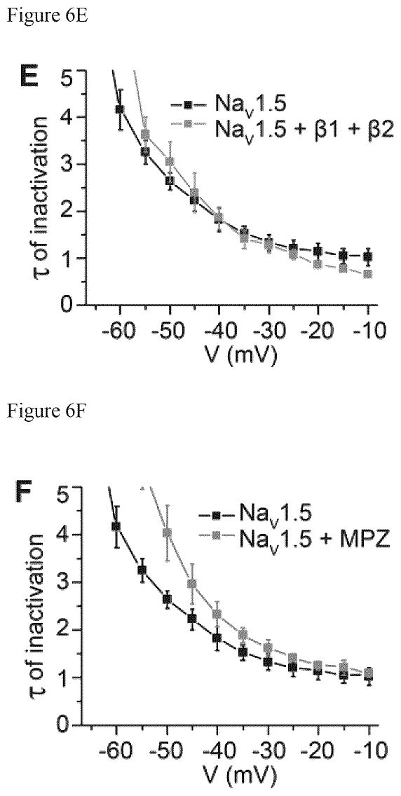

Co-expression of β-subunits can also affect the kinetics of fast-inactivation. In BeHAPe cells, Na_{V}1.5 expressed alone produced a voltage-gated sodium current with fast inactivation that was relatively slow (1.8 ms). Na_{V}1.5 fast inactivation was accelerated by β1 (FIGS. 6A-6F and Table 1), but not by β2 or β4. β3 slowed fast-inactivation at voltages <−45 m V but had no effect at more positive potentials. Surprisingly, expression of MPZ markedly slowed inactivation rates, especially at more negative voltages (<−40 mV).

Lastly, the effect was examined of Na_{V}1.5 currents upon co-expression of both β1 and β2, two β-subunits, which when individually expressed had polar effects on Na_{V}1.5 currents. Expressing both β1 and β2 produced a current that did not inactivate at significantly different rates from Na_{V}1.5 alone yet exhibited the positive shift in the midpoint of the GV and SSI curves that were observed for β1 co-expression alone (9.3±0.6 mV shift in GV, 13.4±0.5 mV shift in SSI) (FIGS. 5A-5F and 6A-6F, and Table 1). These data indicated the effects of β1 override β2 on equilibrium gating, but the effects of β2 dominate the effects of β1 on inactivation kinetics.

### Discussion

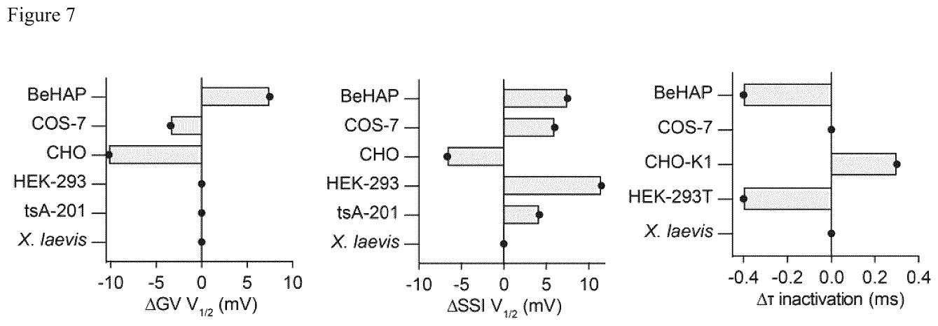

Na_{V}s produce electrical signals in excitable cells, and dysfunction in the channel's pore-forming α-subunit or auxiliary β-subunits are associated with a range of conduction diseases. β-subunits modulate the gating properties of α-subunits. Despite its importance, this function is difficult to study with currently available expression systems. Notably, the effects of β-subunits vary across expression systems as exemplified in Table 2 and FIG. 7.

TABLE 2
Gating properties of Na_{V}1.5 in previous studies

| | | | | | | τ inactivation | |
|---|---|---|---|---|---|---|---|
| | | GV | | SSI | | (ms at −40 mV) | |
| Expression | | V_{1/2} | ΔV_{1/2} | V_{1/2} | ΔV_{1/2} | τ | Δτ |
| System | Channel | (mV) | (mV) | (mV) | (mV) | (ms) | (ms) |
| X. laevis | Na_{V}1.5 | −40.6 ± 1.2 | | −69.2 ± 0.8 | | 0.7 ms | |
| | Na_{V}1.5 + β1 | −40.6 ± 1.9 | NS | −70.5 ± 0.8 | NS | 0.7 ms | NS |
| X. laevis | Na_{V}1.5 | −18.6 ± 4.2 | | −65.3 ± 0.9 | | NR | |
| | Na_{V}1.5 + β1 | −23.3 ± 2.0 | NS | −65.9 ± 0.7 | NS | NR | |
| | Na_{V}1.5 + β3 | −21.3 ± 2.4 | NS | −60.7 ± 1.3 | 5.2 | NR | |
| tsA-201 | Na_{V}1.5 | −25.6 ± 1 | | −77.1 ± 0.5 | | NR | |
| | Na_{V}1.5 + β1 | −24.9 ± 1 | NS | −72.9 ± 1.0 | 4.2 | NR | |
| | Na_{V}1.5 + β2 | −23.2 ± 1 | 2.4 | −77.1 ± 1.1 | NS | NR | |
| | Na_{V}1.5 + | −24.3 ± 2 | NS | −74.0 ± 1.0 | 3.1 | NR | |
| | β1 + β2 | | | | | | |
| tsA-201 | Na_{V}1.5 | −35.4 ± 1.2 | | −88.2 ± 0.9 | | NR | |
| | Na_{V}1.5 + β1 | −34.7 ± 1.2 | NS | −81.8 ± 0.9 | NS | NR | |
| HEK-293T | Na_{V}1.5 | −37.8 ± 1.6 | | −95.2 ± 2.9 | | 2.3 | |
| | Na_{V}1.5 + β1 | −36.7 ± 0.6 | NS | −90.9 ± 2.1 | 4.3 | 1.9 | −0.4 |
| HEK-293T | Na_{V}1.5 | −43.78 ± 1.28 | | −78.77 ± 1.11 | | 0.68 (−20 mV) | |
| | Na_{V}1.5 + β4 | −43.69 ± 1.61 | NS | −82.28 ± 0.74 | −3.5 | 0.77 (−20 mV) | NS |
| HEK-293 | Na_{V}1.5 | NR | | −70.2 ± 1.3 | | NR | |
| | Na_{V}1.5 + β1 | −22 | | −58.7 ± 1.2 | 11.5 | 1.1 ms | |
| CHO | Na_{V}1.5 | −38.7 ± 0.5 | | −86.9 ± 1.5 | | NR | |
| | Na_{V}1.5 + β1 | −48.9 ± 0.8 | −10.2 | −93.6 ± 0.8 | −6.7 | NR | |
| | Na_{V}1.5 + β2 | −38.0 ± 0.6 | NS | −80.2 ± 0.6 | 6.7 | NR | |
| CHO-K1 | Na_{V}1.5 | −16.2 ± 1.0 | | −61.7 ± 1.9 | | 2.0 ms | |
| | Na_{V}1.5 + β1 | −22.2 ± 1.8 | −6 | −68.7 ± 2.5 | −6.7 | 2.3 ms | 0.3 |
| COS-7 | Na_{V}1.5 | −33.5 ± 0.3 | | −95.8 ± 0.3 | | 1.2 ms | |
| | Na_{V}1.5 + β1 | −36.1 ± 0.6 | −3.4 | −89.8 ± 0.2 | 6 | 1.1 ms | NS |

Values are presented as mean ± SEM.

It was hypothesized that the effects of ectopically expressed β-subunits could be better resolved in an expression system without endogenous β-subunits and their phylogenetic relatives, the MPZ-related proteins. Using BeHAPe cells, properties of the main cardiac Na_{V}1.5 channel was observed in the presence and absence of β-subunits. It is important to note that the rate of inactivation and XXX in BeHAPe cells than the eHAP parental counterpart with a full complement of β/MPZ family genes, confirming the premise that these endogenous genes obscure a detailed analysis of β-subunit effects. These findings demonstrate the benefit of using expression systems devoid of endogenous β-subunits, such as BeHAPe cells, in basic studies on α/β interactions. This strategy may also be beneficial in studies on Na_{V} pharmacology and disease-causing mutations.

The studies with Na_{V}1.5 in the presence of single β-subunits revealed that β-subunits have the potential to uniquely alter channel gating. β-subunits displayed various changes in the GV, SSI, and kinetics of inactivation. The results reported here were compared with similarly conducted studies in different expression systems (Table 2). Generally, the BeHAPe cells captured many more β-subunit effects than observed previously. Notably the effects of β1 on the voltage dependence of activation have not been observed in X. laevis oocytes or HEK-293 cells and the effects of voltage dependence of inactivation have not been observed in X. laevis oocytes. It is possible that these differences are due to the presence of endogenous subunits in X. laevis oocytes or HEK-293 cells. Some of the observations hint at the complications that can arise if there is background expression of β-subunits. For example, co-expression of β1 and β2 produced a channel with hybrid properties whereby, some of the effects of individual subunits were overridden. It was also found that the presence of a structurally related protein like MPZ changed Na_{V}1.5 gating dynamics. Thus, studies on β-subunits benefit from removing the whole β/MPZ family.

The effect of β1-β4 on Na_{V}1.5 gating properties reported here could play a major role in cardiac rhythm. Although the specific function of β-subunits in heart are not defined, they are physiologically important as mutations in the subunits are associated with arrhythmias, and knock out of β-subunits (β1-β3) in mice cause cardiac arrythmia. Intriguingly the β-subunits are reported to occupy different subcellular regions in cardiac myocytes and concentrate in different regions of the heart. It seems likely that β-subunits operate in vivo to fine tune heart physiology by modulating Na_{V}1.5 gating at different locales. Another intriguing property of Na_{V}1.5 revealed here is that the inactivation kinetics of the a-subunit are accelerated by only β1. Perhaps this property is linked to forms of heart failure that are accompanied by the persistence of a late sodium current caused by altered inactivation kinetics. If so, altered association with β-subunits within a subpopulation of Na_{V}1.5 complexes might contribute to such a late current.

Despite its clear association with β-subunits, the structural basis for association and modulation of the Na_{V}1.5 α-subunit remains unknown. Interestingly β-subunits most similar in sequence exerted similar effects. β1 and β3 right shift the GV and SSI to more positive voltages, whereas β2 and β4 did not. MPZ, which is similar in sequence to β1 and β3, also shifted the GV and SSI more positive. The effect of MPZ on NaV1.5 is not physiological in the heart since MPZ is not expressed there. However, the effect of MPZ could be used as a structural tool for gaining insight into the molecular basis of the differential effects of the β-subunits. Moreover, other MPZ/β-subunit members such as MPZL2 and MPZL3 are expressed in cardiac myocytes and SCN5A is expressed in other cells besides cardiac myocytes suggesting a potential for Na_{V}1.5 regulation by a wider variety of MPZ/β-subunits. The broader expression pattern of the wider β/MPZ-subunit family and the potential for altering the behavior of other Na_{V} isoforms provides further potential for regulation that remains to be explored.

The effect of combining β1 and β2 was also examined. The combination of β1 and β2 modulated Na_{V}1.5 in ways that were distinct from the effects of individual subunits (Table 1). Some Na_{V} α-subunit isoforms are known to be capable of binding multiple β-subunits simultaneously. Na_{V}1.5 may have this property as well, allowing it to form a hybrid complex with different properties. Further experiments in a cell background such as the BeHAPe cells now enable the nuanced regulatory effects of β-subunits to be dissected.

The strategy for studying the β-subunit family can be employed for characterizations of other gene families. Genes within a family, such as Na_{V} β/MPZ-subunit family, can often compensate for each other due to their structural similarity. In these circumstances, a gene-of-interest might only reveal its full range of activity in an expression system devoid of all compensating isoforms. While deletion of multiple genes from an expression system was previously time consuming, it was shown that it can be done relatively efficiently in haploid eHAP cells. The disadvantage of transfecting multiple gRNAs simultaneously is that recovered clones may have off-target mutations, such that BeHAPe cells may not be precisely congenic with its parent. For this reason, a gene-of-interest should always be characterized by reintroducing it into the new cell line, rather than by comparing the mutant cell line to the wild type parental cells.

Besides Na_{V}1.5, there are nine other α-subunits, and each may be modulated differently by the β-subunits. There are also disease-causing mutations in α and β-subunits that may affect channel properties. The studies in BeHAPe cells demonstrate that the consequences of these interactions are best defined in a clean β/MPZ null background. However, as the BeHAPe cells are only suited to low throughput analysis, a systematic high-throughput electrophysiology analysis would greatly benefit from the engineering of a robust β/MPZ null cell line.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. When used in this specification and the claims as an adverb rather than a preposition, “about” means “approximately” and comprises the stated value and every non-negative value within 10% of that value; in other words, “about 100%” includes 90% and 110% and every value in between.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless stated otherwise, every range or interval includes both endpoints and every value in between.

The invention has been described as “comprising” certain steps and/or elements, which those of skill in the art also “consist of” or “consist essentially of” those steps and/or elements. As used herein, the transitional term “comprising” is synonymous with “including,” “containing,” or “characterized by,” is inclusive or open-ended and does not exclude additional, unrecited elements or method steps. Where the invention is intended to be more narrowly defined, the terms “consisting of” or “consisting essentially of” also are used to describe the invention. As used herein, the transitional phrase “consisting of” excludes any element, step, or ingredient not specified in the claim. The transitional phrase “consisting essentially of” limits the scope of a claim to the specified elements or steps and those that do not materially affect the basic and novel characteristics of the claimed invention. The transitional phrase “consisting essentially of” limits the scope of a claim to the specified materials or steps and those that do not materially affect the basic and novel characteristic(s) of the claimed invention. As used herein, a claim reciting “consisting essentially of” occupies a middle ground between closed claims reciting a “consisting of” format and fully open claims that recite “comprising.” All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for conducting the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A beta/MPZ family-subunit-eliminated (beta/MPZΔ) engineered-cell comprising a cell with functionally inactivated SCN1B, SCN2B, SCN3B, SCN4B, MPZL1, MPZL2, MPZL3, MPZ and JAML genes.

2. The beta/MPZΔ cell of claim 1, wherein the inactivating mutations are stable mutations.

3. The beta/MPZΔ cell of claim 1, wherein all copies of the genes are mutated.

4. The beta/MPZΔ cell of claim 1, wherein the cell is devoid of normal SCN1B, SCN2B, SCN3B, SCN4B, MPZL1, MPZL2, MPZL3, MPZ and JAML expression.

5. The beta/MPZΔ cell of claim 1, wherein the cell is a haploid cell.

6. The beta/MPZΔ cell of claim 1, wherein the cell is a diploid cell.

7. The beta/MPZΔ cell of claim 1, wherein the cell is a cultured immortalized cell.

8. The beta/MPZΔ cell of claim 1, wherein the cell is mammalian cell.

9. The beta/MPZΔ cell of claim 8, wherein the cell is a human cell.

10. The beta/MPZΔ cell of claim 8, wherein the cell is a mouse cell.

11. The beta/MPZΔ cell of claim 8, wherein the cultured immortalized cell is a HEK293, Cos7, or CHO cell.

12. The beta/MPZΔ cell of claim 1, further comprising a defined engineered voltage-gated sodium channel, wherein the engineered voltage-gated sodium channel comprises a sodium channel alpha subunit, at least one sodium channel beta subunit.

13. The beta/MPZΔ cell of claim 12, wherein the defined engineered the sodium channel beta subunit is encoded by SCN1B, SCN2B, SCN3B, or SCN4B.

14. The beta/MPZΔ cell of claim 12, wherein the defined engineered voltage-gated sodium channel further comprises a MPZ family protein.

15. The beta/MPZΔ cell of claim 14, wherein the sodium channel MPZ family protein subunit is encoded by MPZ, MPZL1, MPZL2 or MPZL3.

16. The beta/MPZΔ cell of claim 12, wherein the defined engineered voltage-gated sodium channel further comprises a JAML family protein.

17. A method of screening effectiveness of a drug targeting a voltage-gated sodium channel (VGSC) disease or disorder, comprising
(a) culturing the beta/MPZΔ cell of clam 12,
(b) introducing a target drug to the cells,
(c) applying a high-throughput patch-clamping to the cells to measure gating properties of the cells in the presence of the drugs as compared to control cells; and
(d) comparing the gating properties of the beta/MPZΔ cell the gating properties of the controls cell.

18. The method of claim 17, wherein the VGSC disease or disorder is Long QT syndrome, Brugada syndrome, Atrial standstill, Atrial fibrillation, Sudden infant death syndrome, Sick sinus syndrome, Dilated cardiomyopathy, Generalized epilepsy, Dravet syndrome, Migraine, Autism, Ataxia, Neonatal-infantile seizures, Familial primary erythromelalgia, Hyperkalemic periodic paralysis, Congenital myotonia, or a pain disorder.


SEQUENCE LISTING (81 sequences)

SEQ ID NO: 1 - DNA, 25 bp, synthetic construct
  source 1..25: mol_type = other DNA
agccatggggaggctgctggcctta

SEQ ID NO: 2 - DNA, 102 bp, synthetic construct
  source 1..102: mol_type = other DNA
ccctcaacgtcctcaatggctctgacgcccgcctgccctgcaccttcaactcctgctacacagtgaaccacaaacagttctccctgaactggacttaccagg

SEQ ID NO: 3 - DNA, 86 bp, synthetic construct
  source 1..86: mol_type = other DNA
cccatgaagctgcgctgcatctcctgcatgaagagagaggaggtggaggccaccacggtggtggaatggttctacaggcccgaggg

SEQ ID NO: 4 - DNA, 64 bp, synthetic construct
  source 1..64: mol_type = other DNA
agagaacaggactatgcccggggctggggacggaggcaaagccccggcgagatggctgggcact

SEQ ID NO: 5 - DNA, 23 bp, synthetic construct
  source 1..23: mol_type = other DNA
accgacatctacgctgtcaatgg

SEQ ID NO: 6 - DNA, 73 bp, synthetic construct
  source 1..73: mol_type = other DNA
ccaggccatcgtggtttacaccgacagggaggtccatggtgctgtgggctcccgggtgaccctgcactgctcc

SEQ ID NO: 7 - DNA, 93 bp, synthetic construct
  source 1..93: mol_type = other DNA
ccaggccatcgtgaccctgcactgctccttctggtccagtgagtgggtctcagatgacatctccttcacctggcgctaccagcccgaaggggg

SEQ ID NO: 8 - DNA, 59 bp, synthetic construct
  source 1..59: mol_type = other DNA
atgtatggcaagagctctactcgtgcggtgcttcttctccttggcatacagctcacagg

SEQ ID NO: 9 - DNA, 27 bp, synthetic construct
  source 1..27: mol_type = other DNA
ggcctgtagaaccattccaccacctcc

SEQ ID NO: 10 - DNA, 23 bp, synthetic construct
  source 1..23: mol_type = other DNA
gctgccgggatgcagcagagagg

SEQ ID NO: 11 - DNA, 23 bp, synthetic construct
  source 1..23: mol_type = other DNA
ccacacagtatcagtaagtgtgt

SEQ ID NO: 12 - DNA, 23 bp, synthetic construct
  source 1..23: mol_type = other DNA
cagacagccggcgctggctgtgg

SEQ ID NO: 13 - DNA, 23 bp, synthetic construct
  source 1..23: mol_type = other DNA
tggcaagagctctactcgtgcgg

SEQ ID NO: 14 - DNA, 23 bp, synthetic construct
  source 1..23: mol_type = other DNA
taaggccagcagcctccccatgg

SEQ ID NO: 15 - DNA, 23 bp, synthetic construct
  source 1..23: mol_type = other DNA
ctcctgcatgaagagagaggagg

SEQ ID NO: 16 - DNA, 23 bp, synthetic construct
  source 1..23: mol_type = other DNA
ggagcagtgcagggtcacccggg

SEQ ID NO: 17 - DNA, 23 bp, synthetic construct
  source 1..23: mol_type = other DNA
tacgccaaaagaaatcttcgtgg

SEQ ID NO: 18 - DNA, 23 bp, synthetic construct
  source 1..23: mol_type = other DNA
ttcaagtctactagtacgactgg

SEQ ID NO: 19 - DNA, 20 bp, synthetic construct
  source 1..20: mol_type = other DNA
ctccttggcatacagctcac

SEQ ID NO: 20 - DNA, 23 bp, synthetic construct
  source 1..23: mol_type = other DNA
cgtgcggtgcttcttctccttgg

SEQ ID NO: 21 - DNA, 23 bp, synthetic construct
  source 1..23: mol_type = other DNA
acacacttactgatactgtgtgg

SEQ ID NO: 22 - DNA, 23 bp, synthetic construct
  source 1..23: mol_type = other DNA
tttgaaggtcatcccatacacgg

SEQ ID NO: 23 - DNA, 23 bp, synthetic construct
  source 1..23: mol_type = other DNA
gtagaaccattccaccaccgtgg

SEQ ID NO: 24 - DNA, 23 bp, synthetic construct
  source 1..23: mol_type = other DNA
gaatggttctacaggcccgaggg

SEQ ID NO: 25 - DNA, 23 bp, synthetic construct
  source 1..23: mol_type = other DNA
cctgtcggtgtaaaccacgatgg

SEQ ID NO: 26 - DNA, 20 bp, synthetic construct
  source 1..20: mol_type = other DNA
agtgcagggtcacgatggcc

SEQ ID NO: 27 - DNA, 20 bp, synthetic construct
  source 1..20: mol_type = other DNA
cccttcgggctggtagcgcc

SEQ ID NO: 28 - DNA, 20 bp, synthetic construct
  source 1..20: mol_type = other DNA
tggcgctaccagcccgaagg

SEQ ID NO: 29 - DNA, 20 bp, synthetic construct
  source 1..20: mol_type = other DNA
agagaacaggactatgcccg

SEQ ID NO: 30 - DNA, 20 bp, synthetic construct
  source 1..20: mol_type = other DNA
agtgcccagccatctcgccg

SEQ ID NO: 31 - DNA, 20 bp, synthetic construct
  source 1..20: mol_type = other DNA
accgacatctacgctgtcaa

SEQ ID NO: 32 - DNA, 21 bp, synthetic construct
  source 1..21: mol_type = other DNA
agagctggacggcgacgtaaa

SEQ ID NO: 33 - DNA, 20 bp, synthetic construct
  source 1..20: mol_type = other DNA
aagttcagcgtgtccggcga

SEQ ID NO: 34 - DNA, 20 bp, synthetic construct
  source 1..20: mol_type = other DNA
agcactgcacgccgtaggtc

SEQ ID NO: 35 - DNA, 20 bp, synthetic construct
  source 1..20: mol_type = other DNA
agagccattgaggacgttga

SEQ ID NO: 36 - DNA, 20 bp, synthetic construct
  source 1..20: mol_type = other DNA
ctccctgaactggacttacc

SEQ ID NO: 37 - DNA, 20 bp, synthetic construct
  source 1..20: mol_type = other DNA
taatgatcttcatgcggaac

SEQ ID NO: 38 - DNA, 20 bp, synthetic construct
  source 1..20: mol_type = other DNA
atcctgctgccagtgttact

SEQ ID NO: 39 - DNA, 20 bp, synthetic construct
  source 1..20: mol_type = other DNA
tacccagtaacactggcagc

SEQ ID NO: 40 - DNA, 20 bp, synthetic construct
  source 1..20: mol_type = other DNA
atagactggactctgtcacc

SEQ ID NO: 41 - DNA, 20 bp, synthetic construct
  source 1..20: mol_type = other DNA
gttaccttggcgtgctctcc

SEQ ID NO: 42 - DNA, 20 bp, synthetic construct
  source 1..20: mol_type = other DNA
gggtcatctgagctgtaggc

SEQ ID NO: 43 - DNA, 20 bp, synthetic construct
  source 1..20: mol_type = other DNA
tcctgataacctgcagtggc

SEQ ID NO: 44 - DNA, 20 bp, synthetic construct
  source 1..20: mol_type = other DNA
ttgggacctttgtgcagacg

SEQ ID NO: 45 - DNA, 20 bp, synthetic construct
  source 1..20: mol_type = other DNA
ccggtgttacctgtgtcgtt

SEQ ID NO: 46 - DNA, 20 bp, synthetic construct
  source 1..20: mol_type = other DNA
gggacatggcaggacattca

SEQ ID NO: 47 - DNA, 23 bp, synthetic construct
  source 1..23: mol_type = other DNA
gggaaaactactggctttcaagt

SEQ ID NO: 48 - DNA, 20 bp, synthetic construct
  source 1..20: mol_type = other DNA
ctcccggggacattctaacc

SEQ ID NO: 49 - DNA, 20 bp, synthetic construct
  source 1..20: mol_type = other DNA
cagcaaacctagagccgtca

SEQ ID NO: 50 - DNA, 20 bp, synthetic construct
  source 1..20: mol_type = other DNA
gcactcaccgaaatggcatc

SEQ ID NO: 51 - DNA, 20 bp, synthetic construct
  source 1..20: mol_type = other DNA
ggtggcggagagatcagaag

SEQ ID NO: 52 - DNA, 20 bp, synthetic construct
  source 1..20: mol_type = other DNA
cgcctcgtggtactgacttc

SEQ ID NO: 53 - DNA, 20 bp, synthetic construct
  source 1..20: mol_type = other DNA
cgcaaggtgaaaaagctccc

SEQ ID NO: 54 - DNA, 20 bp, synthetic construct
  source 1..20: mol_type = other DNA
agagctgctgtttctgaccc

SEQ ID NO: 55 - DNA, 20 bp, synthetic construct
  source 1..20: mol_type = other DNA
atgacatcgtcccaacgtcc

SEQ ID NO: 56 - DNA, 20 bp, synthetic construct
  source 1..20: mol_type = other DNA
atttcatgccaaccgtgtgc

SEQ ID NO: 57 - DNA, 20 bp, synthetic construct
  source 1..20: mol_type = other DNA
ctgaccccgctccagatttt

SEQ ID NO: 58 - DNA, 19 bp, synthetic construct
  source 1..19: mol_type = other DNA
cggcggcgactgcagtggc

SEQ ID NO: 59 - DNA, 22 bp, synthetic construct
  source 1..22: mol_type = other DNA
cggtgattgcagccccagacag

SEQ ID NO: 60 - DNA, 19 bp, synthetic construct
  source 1..19: mol_type = other DNA
tctggccaacatggtgaaa

SEQ ID NO: 61 - DNA, 20 bp, synthetic construct
  source 1..20: mol_type = other DNA
aggcacacagttaacattgc

SEQ ID NO: 62 - DNA, 20 bp, synthetic construct
  source 1..20: mol_type = other DNA
gtaggaagaaccagcaaagg

SEQ ID NO: 63 - DNA, 20 bp, synthetic construct
  source 1..20: mol_type = other DNA
gaatgtgtgtgttggggcac

SEQ ID NO: 64 - DNA, 20 bp, synthetic construct
  source 1..20: mol_type = other DNA
aaccggaaaagcgaccatct

SEQ ID NO: 65 - DNA, 20 bp, synthetic construct
  source 1..20: mol_type = other DNA
aattcttacgtggggccctg

SEQ ID NO: 66 - DNA, 20 bp, synthetic construct
  source 1..20: mol_type = other DNA
catcattgccaccgccaaaa

SEQ ID NO: 67 - DNA, 15 bp, synthetic construct
  source 1..15: mol_type = other DNA
ggtcccgccgcctct

SEQ ID NO: 68 - DNA, 20 bp, synthetic construct
  source 1..20: mol_type = other DNA
aacttctgaagctgacttgg

SEQ ID NO: 69 - DNA, 18 bp, synthetic construct
  source 1..18: mol_type = other DNA
cgcacaccttgacaaact

SEQ ID NO: 70 - DNA, 20 bp, synthetic construct
  source 1..20: mol_type = other DNA
tgtacattcagggagcgcag

SEQ ID NO: 71 - DNA, 20 bp, synthetic construct
  source 1..20: mol_type = other DNA
ggagccactctactctggga

SEQ ID NO: 72 - DNA, 20 bp, synthetic construct
  source 1..20: mol_type = other DNA
ctgtagcagctccttgctgt

SEQ ID NO: 73 - DNA, 19 bp, synthetic construct
  source 1..19: mol_type = other DNA
gctaactttcccgagcccc

SEQ ID NO: 74 - DNA, 20 bp, synthetic construct
  source 1..20: mol_type = other DNA
cagctccctgcattgggtaa

SEQ ID NO: 75 - DNA, 20 bp, synthetic construct
  source 1..20: mol_type = other DNA
cccttacctgagcctcctct

SEQ ID NO: 76 - DNA, 20 bp, synthetic construct
  source 1..20: mol_type = other DNA
cacctcacccaggtgttacc

SEQ ID NO: 77 - DNA, 20 bp, synthetic construct
  source 1..20: mol_type = other DNA
acaaggccgttcctaaaggg

SEQ ID NO: 78 - DNA, 20 bp, synthetic construct
  source 1..20: mol_type = other DNA
ggctgggctgtactgttctt

SEQ ID NO: 79 - DNA, 20 bp, synthetic construct
  source 1..20: mol_type = other DNA
cctctgacctgtgactcagc

SEQ ID NO: 80 - DNA, 20 bp, synthetic construct
  source 1..20: mol_type = other DNA
ggctggatgagcaaggaact

SEQ ID NO: 81 - DNA, 18 bp, synthetic construct
  source 1..18: mol_type = other DNA
gggtagctttgtggccga